(19) United States
(12) Patent Application Publication
(10) Pub. No.: US 2024/0425821 A1
(43) Pub. Date: Dec. 26, 2024

(54) CANCER MODELS COMPRISING DENSE TUMOR STROMA

(71) Applicant: THE ADMINISTRATORS OF THE TULANE EDUCATIONAL FUND, New Orleans, LA (US)

(72) Inventors: Mark Justin MONDRINOS, New Orleans, LA (US); Elisabet OLSEN, Memphis, TN (US); Jae MCKEE, New Orleans, LA (US)

(21) Appl. No.: 18/699,533

(22) PCT Filed: Oct. 7, 2022
(86) PCT No.: PCT/US22/77808

§ 371 Date: Apr. 8, 2024

Related U.S. Application Data
(60) Provisional application No. 63/253,522, filed on Oct. 7, 2021.

(51) Int. Cl. C12N 5/09; G01N 33/50

(52) U.S. Cl. CPC C12N 5/0693; G01N 33/5011; C12N 2502/1323; C12N 2503/02; C12N 2513/00; C12N 2533/90

(57) ABSTRACT

The present disclosure relates to cancer tumor stroma models with dense stroma and heterogeneous patterns of ECM anisotropy. The invention herein will enable more accurate modeling of tumor pathophysiology, drug delivery, and novel treatment methods impacted by ECM density.

### TECHNOLOGY FIELD

The present disclosure relates to compositions and systems comprising cancer cells and stromal cells and methods of making. The disclosure also relates to methods of assaying the toxicity or therapeutic effectiveness of an agent on a cancer cell, methods of screening for efficacy of a cancer therapeutic and methods of inducing mechanically activated gene expression in a cell.

### BACKGROUND

Tumor microenvironments play a critical role in the development and progression of cancer as well as its drug resistance capabilities. For example, the detection of a fibrous tumor stroma (a dense tissue containing fibroblasts and collagen fibers) in individuals with non-small cell lung cancer (NSCLC), as well as in other cancers, indicates a poor prognosis for these patients. Collagenous extra cellular matrix (ECM) at lung tumor fronts often exhibit a pattern of concentric layering that creates a barrier to immune cell infiltration. Similar patterns and barriers are present in tumors of other cancers as well. In the field of 3D cancer modeling, current and previous models largely fail to express an accurate stromal density of cancerous tissue due to the average hydrogel model not being nearly as dense as actual tumor stroma.

### SUMMARY

Contractile cells can solve this biomaterials challenge by their ability to contract and deform extracellular matrix (ECM) fibers, which leads to the progressive removal of interstitial fluid. The disclosure relates to compositions comprising human fibroblasts to freely contract collagen type I hydrogels co-seeded with carcinoma cell spheroids to produce a tissue engineered construct with structural features that mimic stroma-rich invasive carcinomas in vivo. In some embodiments, we used a combination of histological, morphometric, mechanical, and genetic analyses to confirm recapitulation of microarchitectural, mechanical, and molecular features of solid tumors with a dense infiltrating stroma. The data of the disclosure establish a paradigm for engineering 3D cancer models with dense stroma that offers user-defined control of the initial cellular and extracellular matrix (ECM) inputs. The physical characteristics of the dense carcinoma constructs engineered using this approach provide a venue for studying the challenges associated with the delivery of macromolecular drugs and cellular immunotherapies to solid tumors. The combination of mechanical and structural mimicry is necessary for accurate modeling of cancer processes relevant to treatment resistance. Drug resistance in cancers has traditionally been attributed to resistant tumor cell phenotypes, which may be intrinsic to driver mutations or may evolve via further mutation and selection pressures imposed by cytotoxic therapies (12). This conceptual model omits the role of physical constraints imposed on therapy by regions of dense and relatively avascular stroma in solid tumors, including longer diffusion distances, more tortuous routes of diffusion in the interstitium, and increased interstitial fluid pressure driving interstitial flow away from the tumor core (6). Similarly, the translation of promising cellular immunotherapies for the treatment of solid tumors is hampered by the inability of cytotoxic T cells to traverse the complex physical barriers in a dense and compacted TME (13, 14). These observations highlight the importance of considering the structural features of solid tumors in the engineering design of 3D cancer models.

Tissue-engineered cancer models that capture challenges to effective therapy imposed by physical constraints of a solid tumor will facilitate and accelerate the pace of front-end hypothesis generating investigations and enhance the accuracy of therapeutic screening studies. Our goal is to recapitulate these heterogeneous structural and mechanical patterns in a tissue engineered system. From a tissue engineering standpoint, first principles dictate that a minimal form of such a model must include a fraction of tumor mass, a fraction of dense collagenous ECM, and a fraction of fibroblasts which are the primary synthesizers of collagen and generators of tension in the ECM via application of contractile force generated by cytoskeletal actomyosin machinery (15).

The disclosure relates to a dense in-vitro tumor model to more accurately predict the success and failure (efficacy or affects) of chemotherapeutics and other cancer therapies on the growth or stability of cancer cells in tumors. By using fibroblasts' intrinsic ability to contract hydrogels loaded with 3D cancer spheroids, we engineered a model of dense tumor stroma that more accurately represents the collagen density seen in these tumors in vivo. This model and its embodiments will enable studies investigating the impairment of cancer therapies such as cellular immunotherapies by dense tumor stroma in an all-human in vitro system. Herein we describe engineering spatially heterogeneous anisotropy in carcinoma constructs based on entrapment of carcinoma cell spheroids which act as solid volume exclusions around which the collagen matrix contracts. This process of tissue contraction and remodeling produces a temporal gradient of increasing ECM density and spatial gradients of ECM density due to local packing and concentric patterns of fiber wrapping around spheroids via a lassoing effect. Structural changes during compaction drive the emergence of mechanical properties that mimic a range of biopsied carcinomas. This easily adaptable method can be performed in any cancer research laboratory using commonly available supplies to produce 3D stroma-rich carcinoma constructs with structural densities and mechanical properties that mimic solid tumors.

In one aspect, the disclosure relates to a composition comprising: (a) a first layer of cells comprising a plurality of cancer cells; (b) a second layer of cells comprising a plurality of stromal cells and extracellular matrix protein; wherein: (i) the density of the extracellular matrix protein is from about 60 milligrams per milliter to about 120 milligrams per milliliter (mg/mL) of volume of cells within a vessel or cell reaction surface; or (ii) the bulk elastic modulus across the first and second layer of cells is from about 6 KPa to about 10 KPa. In some embodiments, the disclosure relates to a composition or system comprising a second layer of cells consist of stromal cells and ECM protein or proteins; and a first layer of cells consisting of cancer cells. In some embodiments, the first layer of cells is present in a spheroid of cancer cells partially or fully enveloped by the second layer of cells. In some embodiments, the second layer of cells consists of fibroblasts. In some embodiments, the ECM protein consists of collagen type I or collagen type III (used interchangeably in this disclosure as Collagen I or Collagen III, respectively).

In some embodiments, the composition further comprises a hydrogel that defines a volume of a vessel within which the plurality of cancer cells and the plurality of stromal cells are positioned, and wherein the hydrogel is absorbed or immobilized to a solid support. In some embodiments, the volume of a vessel is from about 500 microliters to about 6 milliliters in volume, defined by a bottom surface and one or a plurality of side surfaces that create walls around the bottom surface. In some embodiments, the vessel comprises an opening at or proximate to its top region that allows access to the volume of the vessel from a position external to the vessel. In some embodiments, the extracellular matrix protein comprises Collagen I, Collagen III and/or Fibronectin. In some embodiments, the second layer of cells comprises a single extracellular protein chosen from: Collagen I, Collagen III and/or Fibronectin.

The disclosure relates to compositions comprising cells. In some embodiments, the compositions comprise a plurality of cancer cells organized and/or cultured in a spheroid. In some embodiments, the plurality of cancer cells comprise or consist of carcinoma cells. In some embodiments, the spheroid is adjacent to or substantially adjacent to stromal cells and high density extracellular matrix protein. In some embodiments, the stromal cells envelope the cancer cells in a pattern of concentric rings of cells, such that from about 80% to about 100% of the surface area of the spheroid is in contact with stromal cells.

In some embodiments, the extracellular matrix is positioned around the plurality of cancer cells with an elastic modulus of from about 7 KPa to about 9 KPa. In some embodiments, the extracellular matrix is positioned around the plurality of cancer cells at an elastic modulus of about 7 KPa, about 8 KPa, or about 9 KPa. In some embodiments, the stromal cells are at a density from about 400,000 cells per mL of volume to about 1,550,000 cell per mL of volume. In some embodiments, the vessel is about 1 milliter in volume and the stromal cells are seeded in a liquid of about 500,000 cells per milliliter of liquid.

In some embodiments, the plurality of cancer cells are from breast cancer carcinoma cells, from skin cancer carcinoma cells or from prostate cancer carcinoma cells; and/or wherein the plurality of stromal cells comprise fibroblasts. In some embodiments, the carcinoma cells are human cells and the fibroblasts are human cells.

In some embodiments, the plurality of fibroblasts are free of one or more contact points that exert tension on the plurality of cancer cells other than contact with the cancer cells. In some embodiments, a tension exerted on the plurality of cancer cells by the stromal cells is not modulated by contact of the plurality of stromal cells to a point other than the cancer cells. In some embodiments, the system is free of a position within the vessel or culture system upon which the stromal cells are wrapped that exerts a contraction force upon the second layer of cells. In some embodiments, the first layer of cells exerts the only contact point that exerts tension on the second layer of cells.

The disclosure is also directed to a composition comprising: (a) a plurality of stromal cells; (b) a plurality of cancer cells; (c) extracellular matrix protein; and wherein the plurality of cancer cells are in a three-dimensional shape and define a first layer of cells; wherein the plurality of stromal cells are positioned around at least a portion of the cancer cells in a second layer of cells; and wherein the extracellular matrix protein is positioned within the second layer of cells. In some embodiments, the plurality of stromal cells are positioned around the cancer cells in densely packed concentric bundles of anisotrophic cells.

In some embodiments, the extracellular matrix is at a density from about 70 to about 120 mg per mL of volume of the vessel or liquid in which the ECM is positioned into a vessel or vessels.

In some embodiments, the extracellular matrix comprises a thickness of about 10 to about 50 microns around the cancer cells relative to a position proximate to the first layer of cells.

In some embodiments, the second layer of cells are positioned around the cancer cells in anisotropic rings or geometrically similar packing of cells. In some embodiments, the second layer of cells are fibroblasts that are compressed and exhibit geometrically uniform packing around a portion or the entire plurality of cancer cells. In some embodiments, the stromal cells and the cancer cells are in culture for at least about 7 days. In some embodiments, the stromal cells and the cancer cells are in culture from about 7 days to about 24 days. In some embodiments, the bulk elastic modulus of the first and second layer of cells is from about 6 KPa to about 10 KPa. In some embodiments, the extracellular matrix protein comprises one or a combination of Collagen I and/or Collagen III.

In some embodiments, the plurality of cancer cells are carcinoma cells. In some embodiments, the stromal cells are at a density from about 400,000 cells per mL of volume of the vessel to about 1,550,000 cells per mL of volume of the vessel.

In some embodiments, the second layer comprises an anisotropic orientation around the first layer of cells. In some embodiments, the second layer comprises stromal cells in an anisotropic orientation around at least a portion of the first layer of cells such that at least about 50% of the surface area of the cancer cells are in contact with the stromal cells. In some embodiments, the stromal cells are at a density from about 300,000 cells per mL to about 1,550,000 cells per mL of volume of a culture vessel in which the first and second layers are positioned.

In some embodiments, the first layer of cells comprise cells from breast cancer carcinoma cells, skin cancer carcinoma cells, colon carcinoma cells, lung carcinoma cells, and/or prostate cancer carcinoma cells. In some embodiments, the first layer of cells consist of breast cancer carcinoma cells, skin cancer carcinoma cells, colon carcinoma cells, lung carcinoma cells, or prostate cancer carcinoma cells.

In some embodiments, the mass to mass ratio of water content in the first and second layers relative to the total mass of cells and extracellular protein is from about 65% to 75%. In some embodiments, the mass to mass ratio of water content in the first and second layers relative to the total mass of cells and extracellular protein before positioned in culture is from about 65% to 75%.

In some embodiments, the plurality of stromal cells are free of one or more contact points external to the vessel or distal from the cells that exert tension on the second layer cells. In some embodiments, the stromal cells are unconstrained by tension from a point external to the first layer of cells. In some embodiments, the second layer of cells comprises stromal cells that are constrained in tension solely by the cells in the first layer of cells and the surface area of the vessel or cell reactor surface upon which the cells are seeded.

The disclosure is also directed to a system comprising the compositions disclosed herein and a tissue culture media. In some embodiments, the system comprises one or a plurality of vessels positioned on a solid support, each vessel comprising one or a plurality of walls that define a volume into which a first layer of cells and a second layer of cells are positioned. In some embodiments, the first cell layer comprises cancer cells and the second layer comprises stromal cells and extracellular matrix protein. In some embodiments, one or each of the vessels is in a multiplexed format, such that one or a plurality of vessels comprises a first layer and second layer of cells disclosed herein.

In some embodiments, the system further comprises a tissue culture media, the media in contact with at least the second layer of cells. In some embodiments, the tissue culture media is only in contact with a the second layer comprising stromal cells, such that contents of the tissue culture media diffuse through the second layer of cells and into the first layer of cells.

The disclosure also relates to a system further comprising a heating element and a fluid circuit, wherein the fluid circuit comprises an outlet positioned proximate to the one or plurality of vessels, wherein the outlet is in fluid communication with an oxygen and nitrogen source. In some embodiments, the fluid circuit comprises a valve on the oxygen and nitrogen source such that the amount of nitrogen and/or oxygen is adjustable depending upon position of the valve in the fluid circuit. In some embodiments, the system comprises one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet. In some embodiments the cell reactor surface comprise on or a plurality of vessels into which layers of cells are positioned.

The disclosure also relates to a method of assaying the toxicity or therapeutic effectiveness of an agent on a cancer cell comprising: (a) contacting any disclosed composition with the agent. In some embodiments, the method further comprises a step of (b) monitoring the cells for morphologic changes or changes of expression profile of cells after step (a). In some embodiments, the agent is chosen from one or a combination of: an environmental agent, a small molecule therapeutic, a biologic immunotherapy, or a modified T cell. In some embodiments, the agent is a biologic immunotherapy that is an antibody or antibody fragment thereof. In some embodiments, the agent is a modified cell that is a CAR-T cell. In some embodiments, the morphological changes comprise a change in shape, a change in compression or contractility, a change in measure tension across the volume of cells, or a change in gene expression profiling.

The disclosure also relates to a method of screening an agent or library of agents for efficacy as a cancer therapeutic comprising: (a) contacting any disclosed composition with an agent. In some embodiments, the method further comprises a step of (b) observing a change in the cell viability, cell morphology, or cell expression pattern based upon exposure of the agent to the composition. In some embodiments, the step of contacting the composition with an agent comprises contacting the stromal cells, the cancer cells or a combination of both layers of cells with the agent for a time period sufficient for the agent to diffuse throughout the first and second layer of cells. In some embodiments, the the step of contacting the composition with an agent comprises contacting the stromal cells, the cancer cells or a combination of both layers of cells with the agent for a time period sufficient for the agent enter te intracellular compartment of the cells in the first layer and cells in the second layer.

The disclosure also relates to a method of inducing mechanically activated gene expression in a cell comprising culturing any disclosed composition with a tissue culture media. In some embodiments, the method comprises seeding the first and second layer of cells with an extracellular matrix protein or proteins; and allowing the second layer of cells to contract around at least a portion of the cells in the first layer of cells. In some embodiments, the cells are in culture for at least about seven days, about eight days, about nine days, or at least about ten days. In some embodiments, the method further comprises exposing the disclosed composition or compositions to an agent and measuring or observing a change of one or a combination of: (i) extracellular protein density; (ii) tension; (iii) compression; and (iv) packing of cells. In some embodiments, the disclosed methods comprise a step of allowing the second layer of cells to be cultured with the first layer of cells for a time period sufficient to: (i) increase the tension of the second layer of cells around the first layer of cells; (ii) have the second layer of cells compress around the first layer of cells; (iii) the second layer and/or the first layer of cells exhibit increased cell packing as compared to the amount of cell packing in cell not exposed to increased tension (or tension at or below about 5.9 kPa);

The disclosure also relates to a method of inducing mechanically activated gene expression in a cell comprising exposing the disclosed composition to a change of one or a combination of: (i) extracellular matrix protein density; (ii) tension; (iii) compression; and (iv) packing of cells. In some embodiments, the cells are stromal cells and/or cancer cells. In some embodiments, the method further comprises: seeding stromal cells, cancer cells and extracellular matrix protein or proteins in a cell vessel within or on a cell reactor surface before the step of exposing; wherein the step of exposing comprises allowing the stromal cells, constrained by the surface area and volume of the vessel or cell reactor surface, to compress around the cancer cells for a time period to exhibit a change of one or a combination of: (i) extracellular matrix protein density; (ii) tension; (iii) compression; and (iv) packing of cells. In some embodiments, the step of seeding comprising positioning a mixture of cancer cells in a spheroid onto to a reaction surface or vessel comprising a mixture of stromal cells and extracellular matrix protein or proteins. In some embodiments, the stromal cells are human fibroblasts and the extracellular matrix protein is collagen I or collagen III, or a functional fragment thereof. In some embodiments, the second layer of cells consists of human fiberblasts and either collagen I or collagen III, or a functional fragment thereof. In some embodiments, the method further comprises detecting the presence, absence or quantity of mRNA or protein expression of a biomarker in the stromal cells and/or cancer cells; and correlating the presence, absence or quantity of a biomarker with the mechanical activation of the biomarker if the presence, absence or quantity of the biomarker changes when the cells exhibit a change in any one or combination of: (i) increased extracellular matrix protein density; (ii) tension; (iii) compression; and (iv) packing of cells relative to the quantities of the same metrics before the cells are seeded in culture together. Some embodiments include methods where tension and/or contractibility of the first or second layer of cells induces expression of one or a plurality of biomarkers if the expression increase upon the presence of any one or combination of: (i) increased extracellular matrix protein density; (ii) tension; (iii) compression; and (iv) packing of cells relative to the quantities of the same metrics before the cells are seeded in culture together.

The disclosure further relates to a method of manufacturing a cell culture comprising: (a) seeding a plurality of cancer cells; and (b) seeding a plurality of stromal cells for a time period sufficient for the stromal cells to compress around and form a layer of stroma around at least a portion of the plurality of cancer cells. In some embodiments, steps (a) and (b) are performed simultaneously or nearly simultaneously. In some embodiments, the time period sufficient for compression is no less than about seven days. In some embodiments, the time period sufficient for compression is equivalent to a time period sufficient to deposit extracellular protein density around the plurality of cancer cells equivalent to from about 6 KPa to about 10 KPa. In some embodiments, the method further comprises allowing the cells to divide until there are from about 400,000 cells per milliliter to about 1,000,000 cells per milliliter of volume of cells in a vessel.

In some embodiments, methods of the disclosure comprise a step of mixing stromal cells with at least about 2.0 m/mL of extracellular matrix protein before a step of seeding the cells onto a cell culture vessel or cell reactor surface. In some embodiments, the stromal cells are human fibroblasts. In some embodiments, the extracellular matrix protein consists of collagen I or collagen III. In some embodiments, the methods of the disclosure comprise a step of mixing human fibroblasts cells with at least about 2.5 milligram per mL of extracellular matrix protein in a liquid before a step of seeding the cells onto a cell culture vessel or cell reactor surface. In some embodiments, the cells are further mixed with cancer cells. In some embodiments, the human fibroblasts are mixed with carcinoma cells and collagen I or III, at at least about 2.5 mg/mL of fluid.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1 depicts lung cancer cells with dense tumor stroma. Digital photographs and phase contrast images (5×) on day 1 (Panel A) and day 7 (Panel B). Panel C: Immunohistochemical staining for collagen (red). Cropped inset shows heterogeneous ECM architecture and density in peritumoral (P) and distant (D) locations. Panel D: Quantification of collagen matrix density at peritumoral and distant locations.

FIG. 2 depicts the cell sculpting of collagen hydrogels method applied herein.

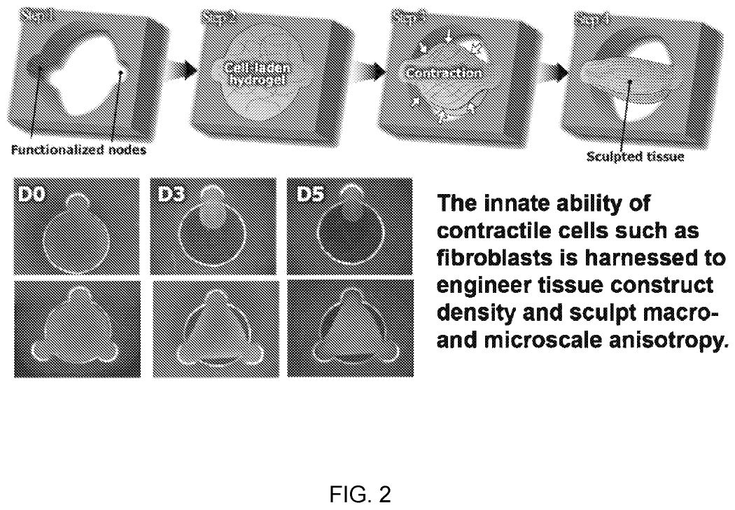

FIG. 3 depicts the application of the cell sculpting concept to engineer 3D tumor models with dense stroma.

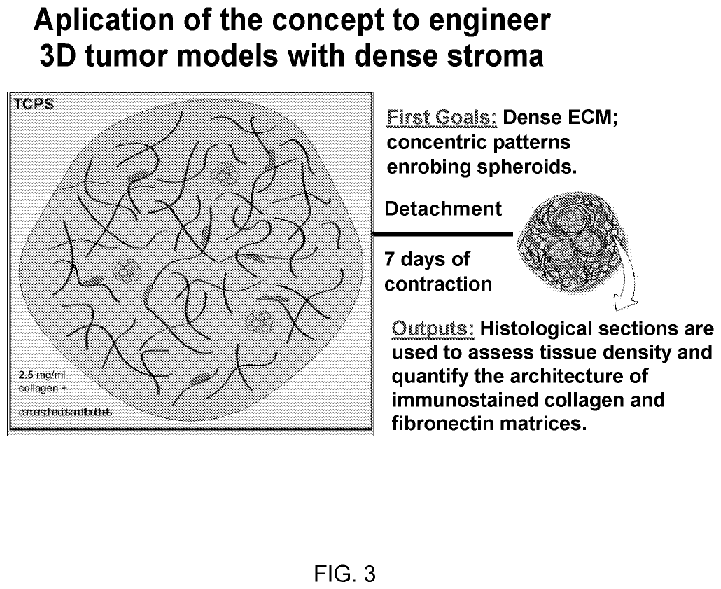

FIG. 4 depicts cell mediated tissue compaction. Digital photographs (top row) and phase contrast micrographs (all images 100×, no cropping) illustrate the rapid volume reduction and increased tissue density by 3 days post-detachment. Further compaction occurs between days 3 and 7. Volume displacement measurements in a graduated microfuge tube indicate that an approximately 20-fold volume reduction occurs (right chart, error bars represent SEM, n=3 constructs). Assuming conservation of collagen mass, this would correlate to a collagen density of ˜50 mg/ml.

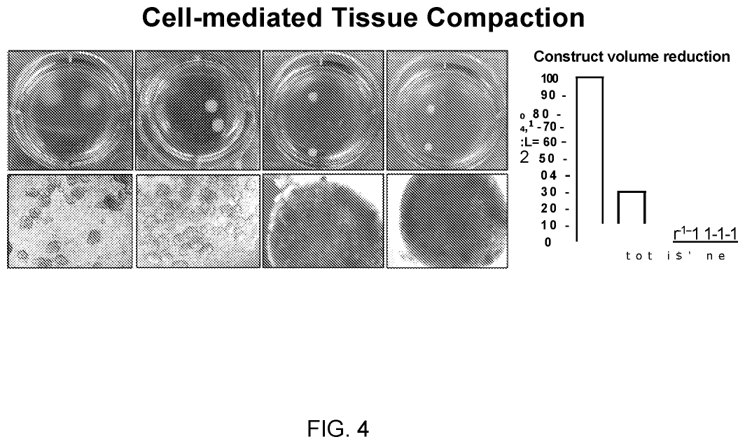

FIG. 5 depicts construct histology. After 7 days, constructs were formalin-fixed and processed for paraffin embedding and sectioning. 5 micron paraffin sections were deparaffinized, rehydrated, and stained with hematoxylin and eosin to visualize tissue histology (left panel). Serial sections were immunostained for the ECM proteins collagen type I (middle panel) and fibronectin (right panel). All images 100×.

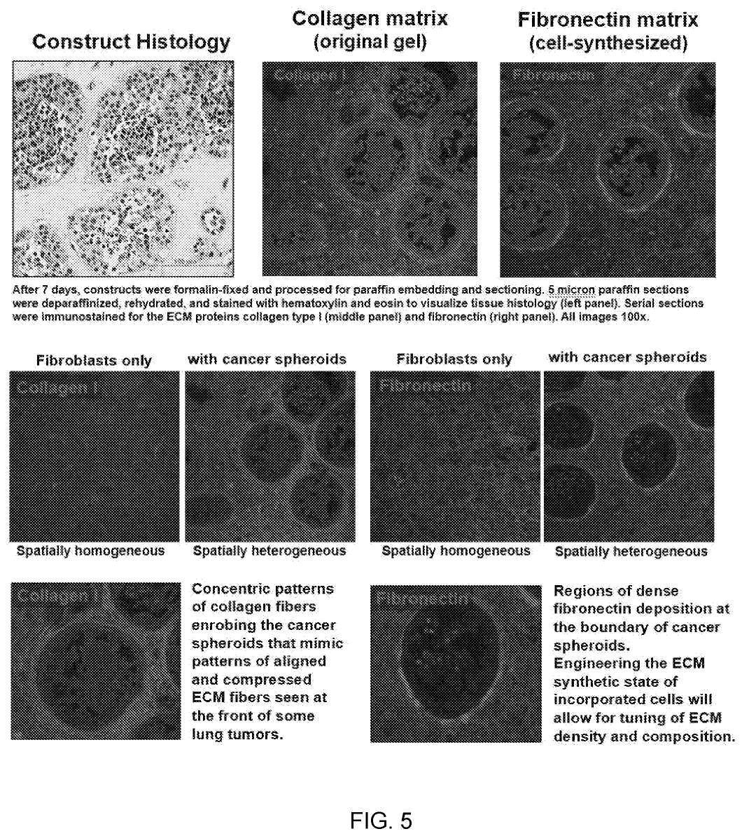

FIG. 6 depicts methods used to quantify ECM architecture using TWOMBLI plugin for FIJI

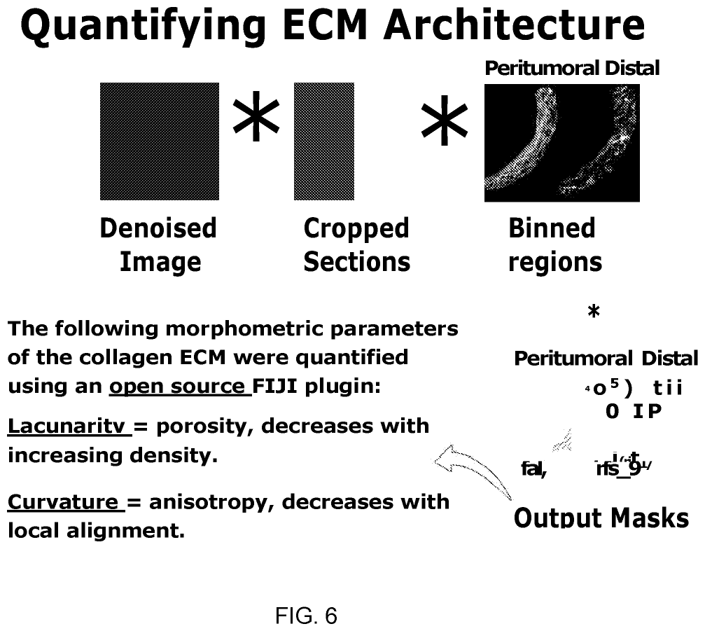

FIG. 7 depicts lacunarity and curvature data generated using the TWOMBLI plugin for FIJI as described above. Error bars represent SEM, n=5.

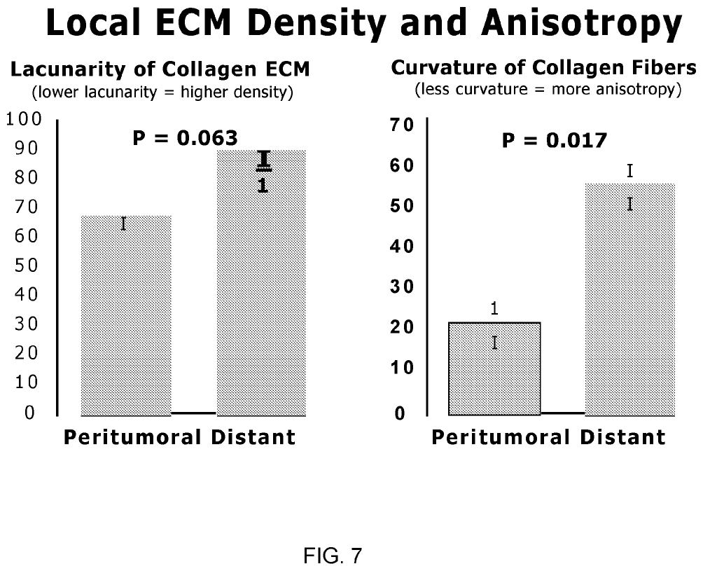

FIG. 8. Harnessing fibroblast contractility to engineer dense carcinoma constructs. (a) Schematic illustration of fibroblast-driven contraction of collagen fibers around cancer spheroids, where blue shapes represent fibroblasts.

FIG. 9. Temporal and spatial gradients of ECM density in dense carcinoma constructs. (9a) H&E stain, (9b) IHC stain for collagen I, and (9c) IHC stain for fibronectin of dense carcinoma construct slices, (9d) One, (9e) four, and (9f) seven-day post-formation IHC collagen I stain of lung carcinoma constructs. Note the increase in collagen I density with time, as illustrated by the above temporal gradient diagram. Day seven (9g) Breast, 9(h) colon, and 9(i) lung carcinoma construct sections with IHC collagen I staining. Note the decrease in collagen I density with distance from the spheroid, as illustrated by the below spatial gradient diagram. 9(j) Normalized mean fluorescent intensity (taken at 10 μm from spheroid) over time of lung carcinoma constructs (n=10). (9k) Normalized mean fluorescent intensity over distance from spheroid of breast, colon, and lung carcinoma constructs (n=10).

FIG. 10. Local anisotropy at tissue interfaces in dense carcinoma constructs. (10a) IHC collagen I stain of dense carcinoma construct slice. Contraction of the gel creates concentric rings of ECM that are oriented around the spheroids. (10b) The collagen fibers become less oriented at farther distances from the spheroid. (10c) This decrease in anisotropy with distance from spheroid is also shown in H&E imaging. (10d) The direction of fibers relative to spheroid tangent line as a function of distance from the spheroid for day one, four, and seven lung carcinoma constructs (n=9). (10e) The direction of fibers relative to the tangent line as a function of distance from the spheroid for day seven lung and colon carcinoma constructs (n=9).

FIG. 11. Progressive tissue construct stiffening driven by fibroblast-mediated contraction. (11a) Image of initial construct (top) and image of construct after seven days (bottom) to illustrate contraction over time. (11b) elastic modulus over time of constructs seeded with 0 and 500K fibroblasts per ml (n=3). (11c) Relationship between density and elastic modulus of constructs.

FIG. 12. Analysis of mechanosensitive gene expression in constrained and unconstrained dense constructs. 12A: Schematic of the two culture configurations. Externally applied tension (blue arrows) from plate adhesions impacts the mechanics of plate bound constructs, counterbalancing cell contractility (red arrows), whereas there is no externally applied tension balancing contractility in the freely contracting constructs. 12B: RT-qPCR gene expression analysis of genes involved in ECM synthesis, fibroblast activation, and contractility. GAPDH was used as a housekeeping gene. Expression levels in the plate-bound constrained constructs were considered the baseline (normalized to a value of 1). Expression in the unconstrained dense constructs is represented as fold change relative to the baseline. N=3 tissues per group. *indicates P<0.05.

FIG. 13. Comparison of carcinoma spheroid invasion in various culture formats. FIG. 13A: Lung carcinoma cell (A549) spheroids in 2.5 mg/ml collagen gel without fibroblasts readily invade the surrounding collagen matrix in plate-bound format by 7 days. FIG. 13B: Lung carcinoma cell (A549) spheroids in 2.5 mg/ml collagen gel with 500,000 fibroblasts/ml readily invade the surrounding collagen matrix in plate-bound format, with the formation of secondary tumor buds from invading cells by 7 days. FIG. 13C: Lung carcinoma cell (A549) spheroids in dense contracted constructs remain spherical with no discernable extensions or secondary buds formed. All images are phase contrast micrographs, original magnification 100×.

### DETAILED DESCRIPTION

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

### Definitions

Unless defined otherwise, technical and scientific terms used herein have the same meaning as commonly understood by one of ordinary skill in the art to which this invention belongs. For example, Singleton et al., Dictionary of Microbiology and Molecular Biology 2nd ed., J. Wiley & Sons (New York, NY 1994), provide one skilled in the art with a general guide to many of the terms used in the present application. Additionally, the practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting.

As used in the present disclosure and claims, the singular forms “a”, “an” and “the” include plural forms unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B.” when used in conjunction with open-ended language such as “comprising” can refer, In some embodiments, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

The term “cancer” as used herein is meant to refer to any disease that is caused by, or results in, inappropriately high levels of cell division, inappropriately low levels of apoptosis, or both. Specific examples of cancer include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. In some embodiments, the cancer cells comprise or consist of one of the above-identified cancer cell types. In some embodiments, the cells are transformed cells that are from one of the above-identified cancer lineages.

By “fragment” is meant a portion of a polypeptide or nucleic acid molecule. This portion contains, preferably, at least 5%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, or 100, 200, 300, 400, 500, 600, 700, 800, 900, 1000 or more nucleotides or amino acids.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “environmental agent” refers to a class of small molecules present in environment that is tested for its capacity to accelerate or inhibit the growth, differentiation or vascularization of cancer cells.

As used herein, the term “small molecule” refers to a low molecular weight (<900 daltons) organic compound that may help regulate a biological process, with a size on the order of 10 9 m. Most drugs are small molecules.

As used herein, the term “biologic immunotherapy” refers to biologic materials such as antibodies, antibody-based fragments, and antibody-drug conjugates.

As used herein, the term “modified T cell” refers to genetically engineered lymphocytes comprising a T-cell receptor on their cell surface.

As used herein, the term chimeric antigen receptor (CAR) T-cells refers to a modified T-cells engineered to target antigens expressed on cancer cells.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The term “culture vessel” as used herein is defined as any vessel suitable for growing, culturing, cultivating, proliferating, propagating, or otherwise similarly manipulating cells. A culture vessel may also be referred to herein as a “culture insert”. In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. In some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated stromal cells. In some embodiments, the culture vessel comprises a hydrogel and one or a plurality of isolated stromal cells, to which one or a plurality of cancer cells are seeded.

The term “exposing” as used herein refers to bringing a disclosed compound and a cell, target receptor, or other biological entity together in direct or indirect contact, in such a manner that the compound can affect the activity of the cell (e.g., receptor, cell, etc.). Directly this can occur by physical contact between the disclosed compound and the cell, receptor o other entity; i.e., by interacting with the target or cell itself, or indirectly this can occur by interacting with another molecule, co-factor, factor, or protein on which the activity of the cell is dependent. In some embodiments, the activity of the cell in response to the compound or molecule is differentiation. In some embodiments, the compound is one or more differentiation factors, therapeutic agents or therapeutic agent candidate.

“Analogues” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like. The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt. As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

As defined herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of the protein in the absence of the inhibitor. In embodiments inhibition refers to reduction of a disease or symptoms of disease. In embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The term “hydrogel” as used herein is defined as any water-insoluble, crosslinked, three-dimensional network of polymer chains with the voids between polymer chains filled with or capable of being filled with water. The term “hydrogel matrix” as used herein is defined as any three-dimensional hydrogel construct, system, device, or similar structure. In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof. In some embodiments, the hydrogel or hydrogel matrix comprises Matrigel® or vitronectin. In some embodiments, the hydrogel or hydrogel matrix can be solidified into various shapes. In some embodiments, the hydrogel or hydrogel matrix comprises poly (ethylene glycol) dimethacrylate (PEG). In some embodiments, the hydrogel or hydrogel matrix comprises Puramatrix. In some embodiments, the hydrogel or hydrogel matrix comprises glycidyl methacrylate-dextran (MeDex). In some embodiments, the hydrogel or hydrogel matrix comprises collagen type I (collagen I) or collagen type III (Collagen III). In some embodiments, two or more hydrogels or hydrogel matrices are used simultaneously cell culture vessel. In some embodiments, two or more hydrogels or hydrogel matrixes are used simultaneously in the same cell culture vessel but the hydrogels are separated by a wall that create independently addressable microenvironments in the tissue culture vessel such as wells. In a multiplexed tissue culture vessel it is possible for some embodiments to include any number of aforementioned wells or independently addressable location within the cell culture vessel such that a hydrogel matrix in one well or location is different or the same as the hydrogel matrix in another well or location of the cell culture vessel.

The term “Matrigel®” means a solubilized basement membrane preparation extracted from the Engelbreth-Holm-Swarm (EHS) mouse sarcoma comprising ECM proteins comprising laminin, collagen IV, heparin sulfate proteoglycans, and entactin/nidogen. In some embodiments, Cultrex® BME (Trevigen, Inc.) or Geltrex® (Thermo-Fisher Inc.) may be substituted for Matrigel®.

In some embodiments, the hydrogel or hydrogel matrices can have various thicknesses. In some embodiments, the thickness of the hydrogel or hydrogel matrix is from about 10 μm to about 3000 μm. In some embodiments, the thickness of the hydrogel or hydrogel matrix is from about 150 μm to about 3000 μm. In some embodiments, the thickness of the hydrogel or hydrogel matrix is from about 200 μm to about 3000 μm. In some embodiments, the thickness of the hydrogel or hydrogel matrix is from about 250 μm to about 3000 μm. In some embodiments, the thickness of the hydrogel or hydrogel matrix is from about 300 μm to about 3000 μm. In some embodiments, the thickness of the hydrogel or hydrogel matrix is from about 350 μm to about 3000 μm. In some embodiments, the thickness of the hydrogel or hydrogel matrix is from about 400 μm to about 3000 μm. In some embodiments, the thickness of the hydrogel or hydrogel matrix is from about 450 μm to about 3000 μm. In some embodiments, the thickness of the hydrogel or hydrogel matrix is from about 500 μm to about 3000 μm. In some embodiments, the thickness of the hydrogel or hydrogel matrix is from about 550 μm to about 3000 μm. In some embodiments, the thickness of the hydrogel or hydrogel matrix is from about 600 μm to about 3000 μm. In some embodiments, the thickness of the hydrogel or hydrogel matrix is from about 650 μm to about 3000 μm. In some embodiments, the thickness of the hydrogel or hydrogel matrix is from about 700 μm to about 3000 μm. In some embodiments, the thickness of the hydrogel or hydrogel matrix is from about 750 μm to about 3000 μm. In some embodiments, the thickness of the hydrogel or hydrogel matrix is from about 800 μm to about 3000 μm. In some embodiments, the thickness of the hydrogel or hydrogel matrix is from about 850 μm to about 3000 μm. In some embodiments, the thickness of the hydrogel or hydrogel matrix is from about 900 μm to about 3000 μm. In some embodiments, the thickness of the hydrogel or hydrogel matrix is from about 950 μm to about 3000 μm. In some embodiments, the thickness of the hydrogel or hydrogel matrix is from about 1000 μm to about 3000 μm. In some embodiments, the thickness of the hydrogel or hydrogel matrix is from about 1500 μm to about 3000 μm. In some embodiments, the thickness of the hydrogel or hydrogel matrix is from about 2000 μm to about 3000 μm. In some embodiments, the thickness of the hydrogel or hydrogel matrix is from about 2500 μm to about 3000 μm. In some embodiments, the thickness of the hydrogel or hydrogel matrix is from about 100 μm to about 2500 μm. In some embodiments, the thickness of the hydrogel or hydrogel matrix is from about 100 μm to about 2000 μm. In some embodiments, the thickness of the hydrogel or hydrogel matrix is from about 100 μm to about 1500 μm. In some embodiments, the thickness of the hydrogel or hydrogel matrix is from about 100 μm to about 1000 μm. In some embodiments, the thickness of the hydrogel or hydrogel matrix is from about 100 μm to about 950 μm. In some embodiments, the thickness of the hydrogel or hydrogel matrix is from about 100 μm to about 900 μm. In some embodiments, the thickness of the hydrogel or hydrogel matrix is from about 100 μm to about 850 μm. In some embodiments, the thickness of the hydrogel or hydrogel matrix is from about 100 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrix comprises one or more synthetic polymers. In some embodiments, the hydrogel or hydrogel matrix comprises one or more of the following synthetic polymers: polyethylene glycol (polyethylene oxide), polyvinyl alcohol, poly-2-hydroxyethyl methacrylate, polyacrylamide, silicones, and any derivatives or combinations thereof.

In some embodiments, the hydrogel or hydrogel matrix comprises one or more synthetic and/or natural polysaccharides. In some embodiments, the hydrogel or hydrogel matrix comprises one or more of the following polysaccharides: hyaluronic acid, heparin sulfate, heparin, dextran, agarose, chitosan, alginate, and any derivatives or combinations thereof.

In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof.

The term “biomarker” as used herein refers to a biological molecule present in an individual or on the surface of a call at varying concentrations useful for determining a phenotype of the cell. A biomarker may include but is not limited to, nucleic acids, proteins and variants and fragments thereof. A biomarker may be DNA comprising the entire or partial nucleic acid sequence encoding the biomarker, or the complement of such a sequence. In some embodiments, the biomarker is an mRNA expression pattern Biomarker nucleic acids useful in the invention are considered to include both DNA and RNA comprising the entire or partial sequence of any of the nucleic acid sequences of interest. In some embodiments, the biomarker is an amino acid sequence expressed upon the presence of a stimulus including any one or combination of: (i) cell density over about 400,000 cells per mL in a vessel; (ii) cell packing; (iii) cell compression; and (iv) tension across the cells that exceeds about 6 kilopascals (KPa). IN some embodiments, the cell packing comprises a structural arrangement of dysmorphic stromal cells that are geometrically uniform and compressed relative to their structural arrangement prior to seeding with a plurality of cancer cells and hydrogel.

The term “two-dimensional culture” as used herein is defined as cultures of cells that lie flat on hydrogels, including Matrigel® and vitronectin, disposed in culture vessels with only a one to four cell height. In some embodiments, two-dimensional culture is not more than 3 cells high. In some embodiments, two-dimensional culture is not more than 2 cells high. In some embodiments, two-dimensional culture is not more than 1 cell high.

As used herein, a “spheroid” or “cell spheroid” means any grouping of cells in a three-dimensional shape that generally corresponds to an oval or circle rotated about one of its principal axes, major or minor, and includes three-dimensional egg shapes, oblate and prolate spheroids, spheres, and substantially equivalent shapes.

A spheroid of the present disclosure can have any suitable width, length, thickness, and/or diameter. In some embodiments, a spheroid may have a width, length, thickness, and/or diameter in a range from about 10 μm to about 50,000 μm, or any range therein, such as, but not limited to, from about 10 μm to about 900 μm, about 100 μm to about 700 μm, about 300 μm to about 600 μm, about 400 μm to about 500 μm, about 500 μm to about 1,000 μm, about 600 μm to about 1,000 μm, about 700 μm to about 1,000 μm, about 800 μm to about 1,000 μm, about 900 μm to about 1,000 μm, about 750 μm to about 1,500 μm, about 1,000 μm to about 5,000 μm, about 1,000 μm to about 10,000 μm, about 2,000 to about 50,000 μm, about 25,000 μm to about 40,000 μm, or about 3,000 μm to about 15,000 μm. In some embodiments, a spheroid may have a width, length, thickness, and/or diameter of about 50 μm, 100 μm, 200 μm, 300 μm, 400 μm, 500 μm, 600 μm, 700 μm, 800 μm, 900 μm, 1,000 μm, 5,000 μm, 10,000 μm, 20,000 μm, 30,000 μm, 40,000 μm, or 50,000 μm. In some embodiments, a plurality of spheroids are generated, and each of the spheroids of the plurality may have a width, length, thickness, and/or diameter that varies by less than about 20%, such as, for example, less than about 15%, 10%, or 5%. In some embodiments, each of the spheroids of the plurality may have a different width, length, thickness, and/or diameter within any of the ranges set forth above.

The cells in a spheroid may have a particular orientation. In some embodiments, the spheroid may comprise an interior core and an exterior surface. In some embodiments, the spheroid may be hollow (i.e., may not comprise cells in the interior). In some embodiments, the interior core cells and the exterior surface cells are different types of cell.

In some embodiments, spheroids may be made up of one, two, three or more different cell types, including one or a plurality of cancer cell types and/or one or a plurality of stem cell types. In some embodiments, the interior core cells comprise one, two, three, or more different cell types. In some embodiments, the interior core cells comprise cancer cells. In some embodiments, the interior core cells comprise carcinoma cells. In some embodiments, the exterior surface cells comprise one, two, three, or more different cell types. In some embodiments, the exterior surface cells comprise stromal cells. In some embodiments, the exterior surface cells comprise fibroblasts.

In some embodiments, the spheroids comprise at least two types of cells. In some embodiments the spheroids comprise stromal cells and cancer cells. In some embodiments, the compositions comprise stromal cells and cancer cells at a ratio of about 5:1, 4:1, 3:1, 2:1 or 1:1 of stromal cells to cancer cells. In some embodiments, the compositions comprise stromal cells and cancer cells at a ratio of about 100:1, 75:1, 50:1, 40:1, 10:1, 5:1, 4:1, 3:1, 2:1 or 1:1. In some embodiments, the compositions comprise cancer cells at a ratio of about 1:5: 1:4, 1:3, or 1:2. Any combination of cell types disclosed herein may be used in the above-identified ratios within the spheroids of the disclosure. In some embodiments, the spheroid consist only of carcinoma cells.

Depending on the particular embodiment, groups of cells may be placed according to any suitable shape, geometry, and/or pattern. For example, independent groups of cells may be deposited as spheroids, and the spheroids may be arranged within a three dimensional grid, or any other suitable three dimensional pattern. The independent spheroids may all comprise approximately the same number of cells and be approximately the same size, or alternatively, different spheroids may have different numbers of cells and different sizes. In some embodiments, multiple spheroids may be arranged in shapes such as an L or T shape, radially from a single point or multiple points, sequential spheroids in a single line or parallel lines, tubes, cylinders, toroids, hierarchically branched vessel networks, high aspect ratio objects, thin closed shells, organoids, or other complex shapes which may correspond to geometries of tissues, vessels or other biological structures. In some embodiments, the compositions comprise spheroids comprising cancer cells wrapped with or covered by a plurality of compressed and highly dense stromal cells. In some embodiments, the stromal cells are constrained in growth only by the surface tension of the cell growth and are free of tension caused by an external point of tension or point of tension other than natural tension exerted by the three-dimensional culture of cells.

The term “subject” as used herein refers to any animal (e.g., a mammal), including, but not limited to, humans, non-human primates, bovines, ovines, porcines, equines, canines, felines, rodents, and the like. In some embodiments, the subject is a mammalian subject. In some embodiments, the subject is a human subject. The terms “subject,” “individual,” and “patient” are used interchangeably herein. The terms “subject,” “individual,” and “patient” thus encompass individuals having disorders such as cancer, for example, carcinoma.

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to treat, combat, ameliorate, prevent or improve one or more symptoms of a viral infection. The activity contemplated by the present methods includes both medical therapeutic and/or prophylactic treatment, as appropriate. The specific dose of a compound administered according to the present disclosure to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds of embodiments of the present disclosure is typically an amount such that when it is administered in a physiologically tolerable excipient composition, it is sufficient to achieve an effective systemic concentration or local concentration in the tissue. In some embodiments, the disclosure relates to identifying a therapeutically effective amount of an agent by exposing the agent to the cells in the disclosed compositions for a time sufficient to modulate cellular activity of the cancer cells or stromal cells, and observing or measuring the changes to the cells in the presence and/or absence of the agent, comparing or normalizing the measurements, and, if the agent inhibits the growth or accelerates the death of the cancer cells at a certain concentration or amount, identifying that concentration or amount as a therapeutically effective amount of the agent.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival of healthy cells as compared to expected survival if not receiving or exposed to the same treatment.

“Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

As used herein, the term “treating cancer” is not intended to be an absolute term. In some aspects, the compositions and methods of the invention seek to reduce the size of a tumor or number of cancer cells, cause a cancer to go into remission, or prevent growth in size or cell number of cancer cells. In some circumstances, treatment with the leads to an improved prognosis.

The term “preventing” or “prevention” or “prevent” as used herein refers to prophylactic or preventative measures that prevent or slow the development of a targeted pathologic condition or disorder. Those in need of treatment include those already diagnosed with the disorder; those prone to have the disorder; and those in whom the disorder is to be prevented.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below; due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence identity to the wild-type sequence upon which the sequence is derived.

By “fragment” is meant a portion of a polypeptide or nucleic acid molecule. This portion contains, preferably, at least about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, or 100, 200, 300, 400, 500, 600, 700, 800, 900, 1000 or more nucleotides or amino acids.

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

“Optional” or “optionally” means that the subsequently described event, circumstance, or material may or may not occur or be present, and that the description includes instances where the event, circumstance, or material occurs or is present and instances where it does not occur or is not present.

### Compositions and Systems

In one aspect, the disclosure relates to a composition comprising: (a) a first layer of cells comprising a plurality of cancer cells; (b) a second layer of cells comprising a plurality of stromal cells and extracellular matrix protein; wherein: (i) the density of the extracellular matrix protein is from about 60 mg/mL to about 120 mg/mL, where the ECM protein is measured in milligrams relative to the total volume of cells in a vessel or the total volume of the vessel into which the cells grow; or (ii) the bulk elastic modulus across the first and second layer of cells is from about 6 KPa to about 10 KPa.

In some embodiments, the first layer of cells is positioned in a spheroid. In some embodiments, the first layer of cells is completely surrounded in three dimensions by the second layer of cells. In some embodiments, there is a “margin” area between the first layer of cells and the second layer of cells comprising a mixture of both cells from the first layer and cells from the second layer.

In some embodiments, the location where the first layer of cells and the second layer of cells are in contact is a tissue interface. In some embodiments, there is local anisotropy at the tissue interface. Anisotropy is a difference, when measured along different axes, in the physical or mechanical properties of the cells. Local anisotropy means that the principal stresses are unequal. In some embodiments, the second layer of cells are positioned around the cancer cells in an anistropic ring of cells.

In some embodiments, the cancer cells are carcinoma cells. In some embodiments, the cancer cells are breast cancer carcinoma cells. In some embodiments, the cancer cells are skin cancer carcinoma cells. In some embodiments, the cancer cells are prostate cancer carcinoma cells. In some embodiments, the cancer cells are lung cancer carcinoma cells. In some embodiments, the cancer cells are colon cancer carcinoma cells. In some embodiments, the cancer cells are sarcoma cells. The disclosure also relates to a composition comprising a vessel or cell reactor surface onto which a first and second layer of cells are positioned; the first layer comprising cancer cells and a second layer comprising stromal cells; wherein the interface between the first and second layers of cells comprises a dense stroma and gradients of increasing ECM density. In some embodiments, the ECM density comprises collagen I or collagen III density of from about 50 to about 150 mg/mL of volume of cells and tissue. In some embodiments, the ECM density comprises collagen I or collagen III density of from about 75 to about 150 mg/ml of volume of cells and tissue. In some embodiments, the ECM density comprises collagen I or collagen III density of from about 100 to about 150 mg/mL of volume of cells and tissue. In some embodiments, the cancer cells are carcinoma cells, the stromal cells are human fibroblasts and the ECM matric comprises or consists of Collagen I at an increasingly density gradient in the direction of the carcinoma cells.

In some embodiments, the stromal cells comprise or consist of fibroblasts. In some embodiments, the stromal cells comprise mesenchymal stem cells. In some embodiments, the stromal cells comprise pericytes. In some embodiments, the stromal cells comprise myofibroblasts. In some embodiments, the stromal cells comprise fibroblast-like stromal cells. In some embodiments, the stromal cells comprise tumor-associated stromal cells. In some embodiments, the stromal cells comprise cell surface markers such as CD44, CD29, CD45, CD105 and/or CD90. In some embodiments, the stromal cells are human fibroblasts.

In some embodiments, the stromal cells and the cancer cells are in culture for at least about 7 days. In some embodiments, the stromal cells and the cancer cells are in culture from about 7 days to about 24 days. In some embodiments the stromal cells and the cancer cells are in culture for at least about 14 days, at least about 21 days, at least about 28 days or more than 28 days.

In some embodiments, the first layer of cells is at least about 100 μm in diameter, at least about 200 μm in diameter, at least about 300 μm in diameter, at least about 400 μm in diameter, at least about 500 μm in diameter, at least about 600 μm in diameter, at least about 700 μm in diameter, at least about 800 μm in diameter, at least about 900 μm in diameter or at least about 1 mm in diameter.

In some embodiments, the second layer of cells is at least about 100 μm in thickness, at least about 200 μm in thickness, at least about 300 μm in thickness, at least about 400 μm in thickness, at least about 500 μm in thickness, at least about 600 μm in thickness, at least about 700 μm in thickness, at least about 800 μm in thickness, at least about 900 μm in thickness or at least about 1 mm in thickness.

In some embodiments, the extracellular matrix protein comprises Collagen I, Collagen III and/or fibronectin.

In some embodiments, the extracellular matrix is positioned within the second layer of cells. In some embodiments, the extracellular matrix is positioned around the plurality of cancer cells with a modulus of from about 7 KPa to about 9 kPa.

In some embodiments, the density of the extracellular matrix protein is from about 60 to about 120 mg/mL. In some embodiments, the density of the extracellular matrix protein is from about 60 to about 110 mg/mL. In some embodiments, the density of the extracellular matrix protein is from about 60 to about 100 mg/mL. In some embodiments, the density of the extracellular matrix protein is from about 60 to about 90 mg/mL. In some embodiments, the density of the extracellular matrix protein is from about 60 to about 80 mg/mL. In some embodiments, the density of the extracellular matrix protein is from about 60 to about 70 mg/mL. In some embodiments, the density of the extracellular matrix protein is from about 70 to about 120 mg/mL. In some embodiments, the density of the extracellular matrix protein is from about 70 to about 110 mg/mL. In some embodiments, the density of the extracellular matrix protein is from about 70 to about 100 mg/mL. In some embodiments, the density of the extracellular matrix protein is from about 70 to about 90 mg/mL. In some embodiments, the density of the extracellular matrix protein is from about 70 to about 80 mg/mL. In some embodiments, the density of the extracellular matrix protein is from about 80 to about 120 mg/mL. In some embodiments, the density of the extracellular matrix protein is from about 80 to about 110 mg/mL. In some embodiments, the density of the extracellular matrix protein is from about 80 to about 100 mg/mL. In some embodiments, the density of the extracellular matrix protein is from about 80 to about 90 mg/mL. In some embodiments, the density of the extracellular matrix protein is from about 90 to about 120 mg/mL. In some embodiments, the density of the extracellular matrix protein is from about 90 to about 110 mg/mL. In some embodiments, the density of the extracellular matrix protein is from about 90 to about 100 mg/mL. In some embodiments, the density of the extracellular matrix protein is from about 100 to about 120 mg/mL. In some embodiments, the density of the extracellular matrix protein is from about 100 to about 110 mg/mL. In some embodiments, the density of the extracellular matrix protein is from about 110 to about 120 mg/mL.

In some embodiments, the extracellular matrix is in a densely packed layer with a thickness of about 10 μm to about 50 μm around the cancer cells relative to a position distal to the first layer of cells. In some embodiments the extracellular matrix is in a densely packed layer with a thickness of about 10 μm to about 40 μm around the cancer cells relative to a position proximate to the first layer of cells. In some embodiments the extracellular matrix is in a densely packed layer with a thickness of about 10 μm to about 30 μm around the cancer cells relative to a position proximate to the first layer of cells. In some embodiments the extracellular matrix is in a densely packed layer with a thickness of about 10 μm to about 20 μm around the cancer cells relative to a position proximate to the first layer of cells. In some embodiments the extracellular matrix is in a densely packed layer with a thickness of about 20 μm to about 50 μm around the cancer cells relative to a position proximate to the first layer of cells. In some embodiments the extracellular matrix is in a densely packed layer with a thickness of about 20 μm to about 40 μm around the cancer cells relative to a position proximate to the first layer of cells. In some embodiments the extracellular matrix is in a densely packed layer with a thickness of about 20 μm to about 30 μm around the cancer cells relative to a position proximate to the first layer of cells. In some embodiments the extracellular matrix is in a densely packed layer with a thickness of about 30 μm to about 50 μm around the cancer cells relative to a position proximate to the first layer of cells. In some embodiments the extracellular matrix is in a densely packed layer with a thickness of about 30 μm to about 40 μm around the cancer cells relative to a position proximate to the first layer of cells. In some embodiments the extracellular matrix is in a densely packed layer with a thickness of about 40 μm to about 50 μm around the cancer cells relative to a position proximate to the first layer of cells.

In some embodiments, the bulk elastic modulus across the first and second layer of cells is from about 6 KPa to about 10 KPa. In some embodiments, the bulk elastic modulus across the first and second layer of cells is from about 6 KPa to about 9 KPa. In some embodiments, the bulk elastic modulus across the first and second layer of cells is from about 6 KPa to about 8 KPa. In some embodiments, the bulk elastic modulus across the first and second layer of cells is from about 6 KPa to about 7 KPa. In some embodiments, the bulk elastic modulus across the first and second layer of cells is from about 7 KPa to about 10 KPa. In some embodiments, the bulk elastic modulus across the first and second layer of cells is from about 7 KPa to about 9 KPa. In some embodiments, the bulk elastic modulus across the first and second layer of cells is from about 7 KPa to about 8 KPa. In some embodiments, the bulk elastic modulus across the first and second layer of cells is from about 8 KPa to about 10 KPa. In some embodiments, the bulk elastic modulus across the first and second layer of cells is from about 8 KPa to about 9 KPa. In some embodiments, the bulk elastic modulus across the first and second layer of cells is from about 9 KPa to about 10 KPa.

Bulk elastic modulus is the ratio of pressure applied to the corresponding relative decrease in the volume of the material. It is represented by the formula:

B=ΔP/(ΔV/V), wherein
B=Bulk Modulus
ΔP=change of the pressure or force applied per unit area on the material
ΔV=change of the volume of the material due to the compression
V=Initial volume of the material
   Bulk elastic modulus can be measured by any method known in the art.

In some embodiments, the composition further comprises a hydrogel.

In some embodiments, the hydrogel is absorbed or immobilized to a solid substrate or solid support. The terms solid substrate or solid support can be used interchangeably and refers to any substance that is free or substantially free of cellular toxins. In some embodiments, the solid substrate comprise one or a combination of silica, plastic, and metal. In some embodiments, a system comprising a cell culture unit is utilized to culture and expand cancer cells described herein, in the presence or absence of stromal cells. In some embodiments, the cell culture unit comprises one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet. In some embodiments, the one or plurality of cell reactor surfaces are configured in a cylindrical form with a hollow volume fixed within a cylindrical first compartment; wherein the first media line and the second media line are positioned on opposite faces of the cylindrical first compartment. The first media line can be attached to a first sealable aperture configured for sterile attachment of a cell culture media source. In some embodiments, the system further comprises a pump and a fluid regulator in operable contact with the first media line, wherein the pump is capable of generating pressure in the first media line and wherein the fluid regulator is capable of regulating a rate of fluid (such as tissue culture media) from a position in a fluid circuit to the pump through the first compartment and into the second media line.

The one or plurality of cell reactor surfaces can have a surface area from about 0.5 m² to about 100.0 m², including any value therein, such as about 3 m², about 4 m², about 5 m², about 6 m², about 7 m², about 8 m², about 9 m², about 10 m², about 11 m², about 12 m², about 13 m², about 14 m², about 15 m², about 16 m², about 17 m², about 18 m², about 19 m², about 20 m², about 21 m², about 22 m², about 23 m², about 24 m², about 25 m², about 26 m², about 27 m², about 28 m², about 29 m², about 30 m², about 31 m², about 32 m², about 33 m², about 34 m², about 35 m², about 36 m², about 37 m², about 38 m², about 39 m², about 40 m², about 41 m², about 42 m², about 43 m², about 44 m², about 45 m², about 46 m², about 47 m², about 48 m², about 49 m², about 50 m², about 51 m², about 52 m², about 53 m², about 54 m², about 55 m², about 56 m², about 57 m², about 58 m², about 59 m², about 60 m², about 61 m², about 62 m², about 63 m², about 64 m², about 65 m², about 66 m², about 67 m², about 68 m², about 69 m², about 70 m², about 71 m², about 72 m², about 73 m², about 74 m², about 75 m², about 76 m², about 77 m², about 78 m², about 79 m², about 80 m², about 81 m², about 82 m², about 83 m², about 84 m², about 85 m², about 86 m², about 87 m², about 88 m², about 89 m², about 90 m², about 91 m², about 92 m², about 93 m², about 94 m², about 95 m², about 96 m², about 97 m², about 98 m², or about 99 m², or about 100 m², or about 105 m².

The system further comprises a gas transfer module in operable connection to the one or plurality of cell reactor surfaces. In some embodiments, the gas module comprises a gas pump and a gas regulator connected to the first compartment by a first gas line. In such embodiments, the first compartment comprises at least one gas outlet. The gas pump is capable of generating air pressure from the pump to the first compartment through the first gas line. The gas outlet can be one or more vents or the gas outlet can be configured for sterile connection to one or more vents. The gas regulator is capable of regulating the speed of gas from the pump through the first compartment.

Some embodiments further comprise a first gas inlet in operable connection to the gas transfer module. In some embodiments, the first gas inlet is attached to a second sealable aperture configured for sterile attachment of a gas source. The gas source can be any known gas storage and/or delivery system, such as for example a container or a tank.

The system can further comprise an apheresis unit in fluid communication with the cell culture unit. Suitable apheresis units include the Spectra Optia Apheresis System (TerumoBCT).

Additionally, in some embodiments, the system further comprises a harvesting compartment in fluid communication with the cell culture unit. Suitable harvesting compartments are discussed elsewhere herein.

A cell culture system as described herein can be used to cancer cells through culturing one or a plurality of stromal cells in the system and allowing the cancer cells and the stromal cells to grow in the first compartment for a time period sufficient to proliferate. In some embodiments, the time period is sufficient to allow the stromal cells to exhibit compaction and grow to a disclosed density around the cancer cells. Cells of the disclosure can be initially introduced into the system and seeded on to the cell reactor surface through on opening to the system's first compartment. After seeding the cells, tissue culture media may be pumped into the system through a fluid circuit that is an open or closed fluid circuit.

The disclosure also relates to a system comprising a cell culture unit comprising one or a plurality of cell reactor surfaces housed in a plurality of compartments, each compartment separated by a removable partition first compartment comprising at least one cell reactor surface, at least one cell reactor surface in fluid connection with a first and second media line, the first media line in fluid communication with a first media inlet, the second media line in fluid communication to a first media outlet. In some embodiments, the cell culture unit comprises a single cell culture chamber comprising multiple partitions, each partition independently removable and independently in fluid connection with the first and the second media line and each partition or set of partitions defining a distinct compartment. In some embodiments, the cell culture unity comprises 2, 3, 4, 5, 6, 7, 8, 9, 10, 11, 12 or more compartments, each compartment separated by and/or defined by one or more partitions. In some embodiments, the compartments are configured in a grid or linear pattern. In some embodiments, each partition separating one compartment from another compartment may be removed such that the cell reactor surface of a first compartment is or becomes contiguous with a cell reactor surface of a second compartment. The removal of one or more partitions allows for an increased surface area onto which cells from one compartment (such as the first compartment) may proliferate and/or grow into another compartment (such as the second compartment) during a method of culturing. In some embodiments, the cell culture unit comprises a set of side walls defining a single surface area divided among 2, 3, 4, 5, 6, 7, 8, 9, 10, 11, 12 or more compartments each compartment with at least one or a plurality of cell reactor surfaces. In some embodiments, each compartment has at least a first cell reactor surface. The disclosure relates to a method of growing T-cell populations on a tissue culture system disclosed herein, wherein primary sets of lymphocytes are plated at about a concentration of from about 0.001 to about 10 million cells per milliliter into one or more compartments of the cell culture unit and then allowed to grow to a confluent layer on surface area of from about 1 to about 200 squared centimeters. In some embodiments, the method further comprises removing one or more partitions to allow the cells to grow in a second compartment until confluence, when again, optionally, another partition may successively be removed to allow for more surface are for expanded culture. In some embodiments the method of culturing further comprises repeating the step of removing a partition for each of the compartments into which cells should grow. In some embodiments, the cell culture unit comprises 3, 4, 5, 6, 7, 8, 9, 10, 11, 12 or more partitions each of which corresponding to the physical barrier between a second and third compartment, between a third and fourth compartment, between a fourth and fifth compartment, between a fifth and sixth compartment, between a sixth and seventh compartment, between a seventh and eighth compartment, between an eighth and ninth compartment, between a ninth and tenth compartment, between a tenth and eleventh compartment, and/or between an eleventh and twelfth compartment, respectively.

In some embodiments, one or more of the partitions comprise an interior portion, a frame portion and an exterior portion. The interior portion of the partition is positioned in the closed portion of the system; the frame portion spans a wall of the culture system separating the interior of the culture system to the exterior of the system; and the exterior portion is positioned outside of the system. In some embodiments, a seal operably fits around the frame portion of one or more of the partitions such that removal of the partition does not introduce pathogens to and/or does not expose the environment outside of the tissue culture system to the interior of the tissue culture system.

In some embodiments, the cell density of each compartment is from about 0.1 to about 10 million cells per mL of cell culture media. In some embodiments, the cell density of each compartment is from about 0.1 to about 10 million cells per mL of cell culture media. In some embodiments, the cell density of each compartment is from about 0.5 to about 10 million cells per mL of cell culture media. In some embodiments, the cell density of each compartment is from about 1.0 to about 10 million cells per mL of cell culture media. In some embodiments, the cell density of each compartment is from about 2 to about 10 million cells per mL of cell culture media. In some embodiments, the cell density of each compartment is from about 3 to about 10 million cells per mL of cell culture media. In some embodiments, the cell density of each compartment is from about 4 to about 10 million cells per mL of cell culture media. In some embodiments, the cell density of each compartment is from about 5 to about 10 million cells per mL of cell culture media. In some embodiments, the cell density of each compartment is from about 6 to about 10 million cells per mL of cell culture media. In some embodiments, the cell density of each compartment is from about 7 to about 10 million cells per mL of cell culture media. In some embodiments, the cell density of each compartment is from about 8 to about 10 million cells per mL of cell culture media. In some embodiments, the cell density of each compartment is from about 9 to about 10 million cells per mL of cell culture media. In some embodiments, the cell density of each compartment is from about 0.1 to about 20 million cells per mL of cell culture media. In some embodiments, the cell density of each compartment is from about 0.1 to about 50 million cells per mL of cell culture media.

In some embodiments, the stromal cells are at a density from about 400,000 cells per mL of volume to about 1,550,000 cells per mL of volume. In some embodiments, the stromal cells are at a density from about 400,000 cells per mL of volume to about 1,500,000 cells per mL of volume. In some embodiments, the stromal cells are at a density from about 400,000 cells per mL of volume to about 1,000,000 cells per mL of volume. In some embodiments, the stromal cells are at a density from about 400,000 cells per mL of volume to about 750,000 cells per mL of volume. In some embodiments, the stromal cells are at a density from about 400,000 cells per mL of volume to about 500,000 cells per mL of volume. In some embodiments, the stromal cells are at a density from about 500,000 cells per mL of volume to about 1,500,000 cells per mL of volume. In some embodiments, the stromal cells are at a density from about 500,000 cells per mL of volume to about 1,250,000 cells per mL of volume. In some embodiments, the stromal cells are at a density from about 500,000 cells per mL of volume to about 1,000,000 cells per mL of volume. In some embodiments, the stromal cells are at a density from about 500,000 cells per mL of volume to about 750,000 cells per mL of volume. In some embodiments, the stromal cells are at a density from about 750,000 cells per mL of volume to about 1,500,000 cells per mL of volume. In some embodiments, the stromal cells are at a density from about 750,000 cells per mL of volume to about 1,250,000 cells per mL of volume. In some embodiments, the stromal cells are at a density from about 750,000 cells per mL of volume to about 1,000,000 cells per mL of volume. In some embodiments, the stromal cells are at a density from about 1,000,000 cells per mL of volume to about 1,500,000 cells per mL of volume. In some embodiments, the stromal cells are at a density from about 1,250,000 cells per mL of volume to about 1,500,000 cells per mL of volume.

In some embodiments, the plurality of fibroblasts are free of one or more contact points that exert tension on the plurality of cancer cells.

In some embodiments, tension exerted on the plurality of cancer cells by the stromal cells is not modulated by contact of the plurality of stromal cells to a synthetic element that creates point of tension exacted on the stromal cells.

The disclosure is also directed to a composition comprising (a) a plurality of stromal cells; (b) a plurality of cancer cells; (c) extracellular matrix protein; and wherein the plurality of cancer cells are in a three-dimensional shape and define a first layer of cells; wherein the plurality of stromal cells are positioned around the cancer cells [in densely packed concentric rings of cells] in a second layer of cells; and wherein the extracellular matrix protein is positioned within the second layer of cells.

### Methods

In some embodiments, the system further comprises one or combination of culture mediums disclosed herein. The disclosure also relates to a method of assaying affect or toxicity of an agent relative to a cancer cell or tumor in vitro, comprising contacting an agent to a layer of stromal cells or a layer of cancer cells disclosed herein. In some embodiments, the method further comprises exposing the one or more cancer cells to an agent. In some embodiments, measuring the one or more morphometric changes comprises measuring morphometry of the one or more cancer cells. The present disclosure also relates to a method of evaluating the toxicity of an agent comprising: (a) culturing one or more cancer cells in any of the compositions described herein; (b) exposing at least one agent to the one or more cancer cells; (c) measuring and/or observing one or more morphometric changes of the one or more cancer cells; and (d) correlating one or more morphometric parameters of the one or more cancer cells with the toxicity of the agent, such that, if the morphometric parameters are indicative of decreased cell viability, the agent is characterized as toxic and, if the morphometric parameters are indicative of unchanged or positive cell viability, the agent is characterized as non-toxic.

In some embodiments, measuring the one or more morphometric changes comprises measuring the compound action potential of the one or more cancer cells.

The present disclosure also relates to a method of inducing growth of one or a plurality of cancer cells in a three dimensional culture vessel comprising a solid substrate, said method comprising: (a) contacting one or a plurality of isolated cancer cells with the solid substrate, said substrate comprising at least one exterior surface, at least one interior surface and at least one interior chamber defined by the at least one interior surface and accessible from a point exterior to the solid substrate through at least one opening; (b) seeding one a plurality of isolated stromal cells to the at least one interior chamber; (c) applying a cell medium into the culture vessel with a volume of cell medium sufficient to cover the at least one interior chamber; wherein the interior chamber comprises a hydrogel. In some embodiments, the stromal cells are fibroblasts, myofibroblasts, mesenychemal cells, or bone cells. In some embodiments, the stromal cells are a single type of stromal cell seeded with at least about 400 thousand, about 500 thousand, about 600 thousand cells. In some embodiments, the stromal cells are a single type of stromal cell seeded with at least about 400 thousand, about 500 thousand, about 600 thousand cells or more and then allowed to be in culture for a time period sufficient for a highly dense ECM protein to form at the interface between the first and second layer of cells a disclosed herein.

In some embodiments, the method further comprises a one or plurality of cancer cells with at least one agent. In some embodiments, the at least one agent is one or a plurality of stem cells or modified T cells. In some embodiments, the T-cells are CAR T cells.

In some embodiments, the at least one agent comprises one or a combination of: laminin, insulin, transferrin, selenium, BSA, FBS, ascorbic acid, type I collagen, and type III collagen. In some embodiments, the at least one agent comprises a small chemical compound. In some embodiments, the at least one agent comprises at least one environmental pollutant.

In some embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

The present disclosure also relates to a method of detecting and/or quantifying cancer cell growth comprising: (a) quantifying one or a plurality of cancer cells; (b) culturing the one or more cancer cells in any of the compositions disclosed herein; and (c) calculating the number of cancer cells in the composition after a culturing for a time period sufficient to allow growth of the one or plurality of cells.

In some embodiments, step (c) comprises detecting an internal and/or external recording of such one or more cancer cells after culturing one or more cancer cells and correlating the recording with a measurement of the same recording corresponding to a known or control number of cells. In some embodiments, the method further comprises contacting the one or more cancer cells to one or more agents. In some embodiments, step (c) comprises measuring an internal and/or external recording before and after the step of contacting the one or more cancer cells to the one or more agents; and correlating the difference in the recording before contacting the one or more cancer cells to the one or more agents to the recording after contacting the one or more cancer cells to the one or more agents to a change in cell number.

The present disclosure also relates to a method of detecting or quantifying of cancer cell growth comprising: (a) quantifying the one or plurality of cancer cells in one or more of the composition disclosed herein; (b) contacting the one or plurality of cancer cells to one or a plurality of agents; and (c) quantifying the amount of biomarker expression in the one or plurality of cells after contacting the one or plurality of cells to one or a plurality of agents; and (d) calculating the difference in the number of cancer cells in culture prior to the step (c) and after step (c). The present disclosure also relates to a method of detecting or quantifying of cancer cell growth comprising: (a) seeding one or a plurality of cancer cells in any of the compositions disclosed herein; (b) quantifying expression of one or more biomarkers in the one or plurality of cancer cells; (c) contacting the one or plurality of cancer cells to one or a plurality of agents; and (d) quantifying one or more biomarkers in the one or plurality of cells after contacting the one or plurality of cells to one or a plurality of agents; and (e) calculating the difference in the number of cancer cells in culture prior to the step (c) and after step (c). In some embodiments, the step of quantifying comprises staining the one or plurality of a cancer cells. In some embodiments, steps (b), (d), and/or (e) are performed via microscopy or digital imaging.

The present disclosure also relates to a method of measuring intracellular or extracellular expression of nucleic acid (e.g. mRNA expression) or expression of a protein biomarker comprising: (a) culturing one or a plurality of cancer cells in any of the composition disclosed herein; (b) allowing the one or a plurality of cancer cells to form a spheroid (c) mixing the cells with stromal cells and ECM protein or proteins; (d) seeding the cells in a vessel or on cell reactor surface; (e) allowing the stromal cells and ECM protein or proteins to form dense anisotrophic tissue around at least a portion or around the entire spheroid; and (f) taking a sample of the stromal cells or the cancer cells; (g) isolating RNA or protein from the sample; and (h) measuring the quantity of biomarkers from the RNA or protein sample. In some embodiments, the step of measuring the quantity of biomarkers comprises conducting RT-PCR or performing immunohistochemistry. In some embodiments, the stromal cells are human fibroblasts and the cancer cells are human carcinoma cells. In some embodiments, the method further comprises exposing the stromal cells and cancer cells to an agent after step (e). In some embodiments, the method further comprises exposing the stromal cells and cancer cells to an agent after step (e); and measuring the quantity of biomarkers from the RNA or protein sample before and after exposing step.

The present disclosure also relates to a method of measuring or quantifying any therapeutic effect of an agent comprising: (a) culturing one or a plurality of cancer cells in any of the composition disclosed herein in the presence and absence of the agent; (b) applying a voltage potential across the one or a plurality of cancer cells in the presence and absence of the agent; or measuring the modulus or tension on the one or plurality of cancer cells and/or stromal cells in the presence and absence of the agent or observing the morphological changes of the stromal cells or cancer cells; and (c) correlating the difference in tension or modulus or change of morphology through the one or plurality of cancer cells to the therapeutic effect of the agent, such that a decline in viability or cell health or a decrease in tension in the presence of the agent as compared to the tension measured or morphology observed in the absence of the agent is indicative of a therapeutic effect and no change or an increased tension or no change in morphology or viability in the presence of the agent as compared to the same measured or observed in the absence of the agent is indicative of the agent not conferring a therapeutic effect.

The present disclosure also relates to a method of detecting or quantifying morphology changes due to the presence, absence or change in the amount of agent exposed to the cells in vitro comprising: (a) culturing one or a plurality of cancer cells and fibroblasts with ECM protein in any of the composition disclosed herein; (b) exposing an agent to the cells; and (c) measuring or observing an effect of the agent on the one or plurality of cancer cells. In some embodiments, the method further comprises correlating a reduced viability of the cancer cells in step (c) to the positive effect of the agent as compared to the same measurements or observations of the cancer cells and stromal cells not exposed to the agent. In some embodiments, the method further comprises observing the cells through imaging the one or plurality of cells with a microscope and/or digital camera.

The present disclosure also relates to a method of culturing a a carcinoma cell in culture comprising: (a) culturing one or a plurality of carcinoma cells in any of the composition disclosed herein; and (b) exposing the cancer cells to an agent in the presence of a layer of stromal cells and ECM material. In some embodiments, the interface between a plurality of carcinoma cells and fibroblasts comprise a gradient of increasing density of ECM material or protein (such as Collagen I and Collagen III) in the direction proximate to the cancer cells. In some embodiments, the methods are free of exertion of tension on the carcinoma cells except the tension created by the second layer of cells (stromal cells).

The present disclosure also relates to a method of measuring or quantifying toxicity of an agent comprising: (a) culturing one or a plurality of cancer cells in any of the composition disclosed herein in the presence and absence of the agent; (b) measuring the Young's or bulk modulus or tension across the one or plurality of cancer cells and/or stromal cells in the presence and absence of the agent or observing the morphological changes of the stromal cells or cancer cells; and (c) correlating the difference in tension or modulus or change of morphology through the one or plurality of cancer cells to toxicity of the agent, such that a decline in viability or cell health or a decrease in tension in the presence of the agent as compared to the tension measured or morphology observed in the absence of the agent is indicative of a toxic effect and no change or an increased tension or no change in morphology or viability in the presence of the agent as compared to the same measured or observed in the absence of the agent is indicative of the agent not conferring a toxic effect.

Other embodiments are described in the following non-limiting Examples. Various publications, including patents, published applications, GenBank Accession Numbers, and technical articles and scholarly articles are cited throughout the specification. Each of these cited publications is incorporated by reference herein in its entirety.

### REFERENCES

1. Barcellos-Hoff M H, Aggeler J, Ram T G, & Bissell M J (1989) Functional differentiation and alveolar morphogenesis of primary mammary cultures on reconstituted basement membrane. Development 105(2):223-235.
2. Petersen O W, Ronnov-Jessen L, Howlett A R, & Bissell M J (1992) Interaction with basement membrane serves to rapidly distinguish growth and differentiation pattern of normal and malignant human breast epithelial cells. Proc Natl Acad Sci USA 89(19):9064-9068.
3. Drost J & Clevers H (2018) Organoids in cancer research. Nat Rev Cancer 18(7):407-418.
4. Bahcecioglu G, Basara G, Ellis B W, Ren X, & Zorlutuna P (2020) Breast cancer models: Engineering the tumor microenvironment. Acta Biomater 106:1-21.
5. Wu J, Liang C, Chen M, & Su W (2016) Association between tumor-stroma ratio and prognosis in solid tumor patients: a systematic review and meta-analysis. Oncotarget 7(42):68954-68965.
6. Weniger M, Honselmann K C, & Liss A S (2018) The Extracellular Matrix and Pancreatic Cancer: A Complex Relationship. Cancers (Basel) 10(9).
7. Kota J, Hancock J, Kwon J, & Korc M (2017) Pancreatic cancer: Stroma and its current and emerging targeted therapies. Cancer Lett 391:38-49.
8. Piersma B, Hayward M K, & Weaver V M (2020) Fibrosis and cancer: A strained relationship. Biochim Biophys Acta Rev Cancer 1873(2):188356.
9. Paszek M J, et al. (2005) Tensional homeostasis and the malignant phenotype. Cancer Cell 8(3):241-254.
10. Ahmadzadeh H, et al. (2017) Modeling the two-way feedback between contractility and matrix realignment reveals a nonlinear mode of cancer cell invasion. Proc Natl Acad Sci USA 114(9):E1617-E1626.
11. Engler A J, Sen S, Sweeney H L, & Discher D E (2006) Matrix elasticity directs stem cell lineage specification. Cell 126(4):677-689.
12. Emran T B, et al. (2022) Multidrug Resistance in Cancer: Understanding Molecular Mechanisms, Immunoprevention and Therapeutic Approaches. Front Oncol 12:891652.
13. Marofi F, et al. (2021) CAR T cells in solid tumors: challenges and opportunities. Stem Cell Res Ther 12(1):81.
14. Lanitis E, Dangaj D, Irving M, & Coukos G (2017) Mechanisms regulating T-cell infiltration and activity in solid tumors. Ann Oncol 28(suppl_12): xii18-xii32.
15. Hinz B, McCulloch C A, & Coelho N M (2019) Mechanical regulation of myofibroblast phenoconversion and collagen contraction. Exp Cell Res 379(1):119-128.
16. Shiga K, et al. (2015) Cancer-Associated Fibroblasts: Their Characteristics and Their Roles in Tumor Growth. Cancers (Basel) 7(4):2443-2458.
17. Bell E, Ivarsson B, & Merrill C (1979) Production of a tissue-like structure by contraction of collagen lattices by human fibroblasts of different proliferative potential in vitro. Proc Natl Acad Sci USA 76(3):1274-1278.
18. Mondrinos M J, et al. (2021) Surface-directed engineering of tissue anisotropy in microphysiological models of musculoskeletal tissue. Sci Adv 7(11).
19. Valkenburg K C, de Groot A E, & Pienta K J (2018) Targeting the tumour stroma to improve cancer therapy. Nat Rev Clin Oncol 15(6):366-381.
20. Kaur A, et al. (2019) Remodeling of the Collagen Matrix in Aging Skin Promotes Melanoma Metastasis and Affects Immune Cell Motility. Cancer Discov 9(1):64-81.
21. Wolf K, et al. (2013) Physical limits of cell migration: control by ECM space and nuclear deformation and tuning by proteolysis and traction force. J Cell Biol 201(7):1069-1084.
22. Swift J, et al. (2013) Nuclear lamin-A scales with tissue stiffness and enhances matrix-directed differentiation. Science 341(6149):1240104.
23. Harn H I, et al. (2019) The tension biology of wound healing. Exp Dermatol 28(4):464-471.
24. Grunert P, et al. (2015) Riboflavin crosslinked high-density collagen gel for the repair of annular defects in intervertebral discs: An in vivo study. Acta Biomater 26:215-224.
25. Hong M, Clubb J D, & Chen Y Y (2020) Engineering CAR-T Cells for Next-Generation Cancer Therapy. Cancer Cell 38(4):473-488.
26. Martinez M & Moon E K (2019) CAR T Cells for Solid Tumors: New Strategies for Finding, Infiltrating, and Surviving in the Tumor Microenvironment. Front Immunol 10:128.
27. Salmon H, et al. (2012) Matrix architecture defines the preferential localization and migration of T cells into the stroma of human lung tumors. J Clin Invest 122(3):899-910.
28. Li M, et al. (2005) Electrospun protein fibers as matrices for tissue engineering. Biomaterials 26(30):5999-6008.
29. Braziulis E, et al. (2012) Modified plastic compression of collagen hydrogels provides an ideal matrix for clinically applicable skin substitutes. Tissue Eng Part C Methods 18(6):464-474.
30. Calderon-Colon X, et al. (2012) Structure and properties of collagen vitrigel membranes for ocular repair and regeneration applications. Biomaterials 33(33):8286-8295.
31. Mondrinos M J, Yi YS, Wu N K, Ding X, & Huh D (2017) Native extracellular matrix-derived semipermeable, optically transparent, and inexpensive membrane inserts for microfluidic cell culture. Lab Chip.
32. Legant W R, et al. (2009) Microfabricated tissue gauges to measure and manipulate forces from 3D microtissues. Proc Natl Acad Sci USA 106(25):10097-10102.
33. Wang H, et al. (2013) Necking and failure of constrained 3D microtissues induced by cellular tension. Proc Natl Acad Sci USA 110(52):20923-20928.
34. Abhilash A S, Baker B M, Trappmann B, Chen C S, & Shenoy V B (2014) Remodeling of fibrous extracellular matrices by contractile cells: predictions from discrete fiber network simulations. Biophys J 107(8):1829-1840.
35. Cross V L, et al. (2010) Dense type I collagen matrices that support cellular remodeling and microfabrication for studies of tumor angiogenesis and vasculogenesis in vitro. Biomaterials 31(33):8596-8607.
36. Kawano S, et al. (2015) Assessment of elasticity of colorectal cancer tissue, clinical utility, pathological and phenotypical relevance. Cancer Sci 106(9):1232-1239.
37. Samani A, Zubovits J, & Plewes D (2007) Elastic moduli of normal and pathological human breast tissues: an inversion-technique-based investigation of 169 samples. Phys Med Biol 52(6):1565-1576.
38. Raub C B, Putnam A J, Tromberg B J, & George S C (2010) Predicting bulk mechanical properties of cellularized collagen gels using multiphoton microscopy. Acta Biomater 6(12):4657-4665.
39. Panciera T, Azzolin L, Cordenonsi M, & Piccolo S (2017) Mechanobiology of YAP and TAZ in physiology and disease. Nat Rev Mol Cell Biol.
40. Au-Liu F & Au-Tschumperlin D J (2011) Micro-Mechanical Characterization of Lung Tissue Using Atomic Force Microscopy. JoVE (54):e2911.
41. van Oosten A S G, et al. (2019) Emergence of tissue-like mechanics from fibrous networks confined by close-packed cells. Nature 573(7772):96-101.

### EXAMPLE 1

### Methods

Cell Culture. Normal human lung fibroblasts (HLFs) [American Type Culture Collection (ATCC) and Lonza] were cultured in fibroblast basal medium with low serum kit (ATCC) and 2% fetal bovine serum (FBS). A549 lung adenocarcinoma cells (ATCC) were cultured in F-12K medium supplemented with 10% FBS. HT-29 colorectal adenocarcinoma cells (ATCC) were cultured in McCoy's 5A supplemented with 10% FBS. MCF-7 breast adenocarcinoma cells were cultured in DMEM or F12K with 10% FBS. All culture media contained 1% antibiotic-antimycotic (Corning) and cultured in a humidified tissue culture incubator at 37° C. with 5% CO2. Typical passage numbers used. Confluency at splits.

Constructing Dense Carcinoma Cells. Cells were harvested and collected using standard trypsinization. A 2.5 mg/ml collagen I hydrogel solution was prepared according to manufacturer protocols (Corning). HLF were introduced into the collagen mixture at a density of 2.5-5.0×105 cells/ml and gel droplets were made by pipetting 50 uL of gel solution onto a 6 well plate. Higher initial cell densities produced more rapid contraction which was not desired for the current study. Dense carcinoma constructs were formed by co-seeding carcinoma cell spheroids (A549, MCF7, or HT29) along with HLF. Carcinoma spheroids were cultured for 5 days in low attachment plates (Corning) prior to tissue construct formation. Constructs were cultured for cultured in the plate-bound format for 24 hours in FGM with 2% FBS. After 24 hours, constructs were manually detached from plate surfaces using a pipet tip or sterilized spatula and allowed to freely contract for 1, 4, or 7 days in the same culture medium prior to collection for end-point analysis.

Immunohistochemistry. Paraformaldehyde-fixed tissues were taken to the Pathology Core Laboratory at Tulane University Health Sciences Center for paraffin embedding and sectioning. Hematoxylin and eosin (H&E) stained sections were provided by the core. Paraffin sections were deparaffinized, rehydrated and processed for immunohistochemical staining using standard procedures. After rehydration, sections were washed with PBS, blocked and permeabilized in PBS containing 3% bovine serum albumin (BSA) and 0.1% Triton X, prepared in PBS for at least 30 min at room temperature, then incubated for 2-3 hours at room temperature with primary antibodies against collagen type I (Abcam, ab34710) or fibronectin (Abcam, abXX) at dilutions of 1:200 in PBS with 1% BSA. After washing, secondary antibodies were incubated at dilution of 1:1000 along with Hoechst dye as a nuclear counterstain in PBS with 1% BSA for 30 minutes at room temperature. After final washes in PBS, slides were mounted with antifade medium (Vector Labs) and stored prior to fluorescence microscopy.

Microscopy and Image Analysis. All slides were imaged on a Nikon Ti-2 Confocal Microscope and images were acquired using a XYZ camera. Collagen density and directional orientation were quantified via analysis of fluorescence micrographs. Images for quantitative analysis were acquired with the same exposure times and laser power settings. Total MFI. For collagen density analysis, fluorescence intensity was measured in FIJI across 30 μm in four regions of interest, starting at a tangent line to the spheroid interface and moving outward along a perpendicular line. ROIs were 50×10 μm with the longer side of each ROI running parallel to the initial tangent of the spheroid. At least 10 sections were imaged analyzed for each experimental group and time point. Collagen directionality analysis was performed using micrographs of H&E stained slides and the FIJI directionality plugin. Orientation of tissue structures labeled by eosin was measured in ROIs extending 45 μm from the spheroid interface. Orientation refers to degrees from the tangent line. Percent goodness of fit of a Gaussian curve to the orientation histogram produced in FIJI was used to assess variance in the data for each ROI. At least 10 sections were imaged analyzed for each experimental group and time point.

Mechanical Testing. A nanotribometer (CETR, UMT Multi-specimen Test System) with the force sensitivity of 1 μN [10] was used to investigate the stress-strain response of the soft gels under ambient conditions.

In a typical embodiment, a gel sample (typically thickness of 0.8-1.2 mm) was placed on a glass slide. Phosphate buffered saline was added to the gel sample to not only preserve the hydration and structural integrity of the tissue construct, but also to minimize the adhesive interaction between the probe and the substrate. A stainless steel cylindrical-shaped rod with a diameter of 1 mm was used as the probe with an approach velocity of 0.065 mm/s. A cylindrical-shaped probe was chosen so as to maintain a constant contact area with the sample's surface thereby minimizing errors associated with adhesive dissipation energy [11]. In all experiments, surface contact and indentation depth were controlled to obtain reproducible test results [12]. Effects of vibrations from the test systems was also dampened by (insert coat) coating to prevent fluctuations in measurements. The initial contact was determined by locating the force increase in the force vs time data. Then the initial linear region of this force vs time data was used to calculate a stress and strain values:

$$σ ( t ) = F z ( t ) / Ap \qquad ( 1 )$$

$$ϵ ( t ) = z ( t ) - z 0 - Fz ( t ) / k * th \qquad ( 2 )$$

where σ(t) is the stress at time t, Fz(t) is the force applied to the gel at time t, Ap is the surface area of the probe, ε(t) is the strain at time t, z(t) is the height of the probe at time t, z0 is the height of the probe when it makes contact with the gel, k is the spring constant, and th is the thickness of the gel. Thickness of the gel was measured in Fiji via micrographs of each gel that were captured by a CAMERA NAME HERE. The resulting stress/strain curve was used to obtain the linear response of material to the compressive stress. A linear regression line was then fit to the curve. The slope of this regression line was equal to the elastic modulus (E) of the gel sample. Six total groups of 50 ml gels were tested (500K HLFs/ml and 0 HLFs/ml for day 1,4, and 7) with six replicates per group. Day 1 refers to the day at which gels were detached from the well plate. Results of the three closest elastic moduli values were selected as representative replicates from each group. All data analysis was performed in R.

### Gene Expression Analysis

Constrained and unconstrained were harvested after 7 days of culture and stabilized in RNALater tissue reagent prior to RNA isolation. Isolation of total RNA from gels was adapted from Qiagen RNeasy manufacturers protocol (Qiagen GmBH, Hilden, Germany), and RNA purity was measured. For cDNA synthesis, 1 μg of RNA was used with qScript cDNA supermix (Quantabio) according to manufacturer's instructions. The final cDNA sample was diluted in a 1:10 ratio in ultrapure water and stored in −20° C. until use. RT-qPCR occurred using Thermofischer Step One Plus instrument and was quantified using SYBR Green Power Up master mix. The primer sequences in Table 1 were used.

TABLE 1
Primer Sequences

| | |
|---|---|
| GAPDH F (SEQ ID NO: 1) | TTAAAAGCAGCCCTGGTGAC |
| GAPDH R (SEQ ID NO: 2) | CTCTGCTCCTCCTGTTCGAC |
| aSMA F (SEQ ID NO: 3) | CCGACCGAATGCAGAAGGA |
| aSMA R (SEQ ID NO: 4) | ACAGAGTATTTGCGCTCCGAA |
| SMAD2 F (SEQ ID NO: 5) | ATGTCGTCCATCTTGCCATTC |
| SMAD2 R (SEQ ID NO: 6) | AACCGTCCTGTTTTCTTTAGCTT |
| MLCK F (SEQ ID NO: 7) | CCCGAGGTTGTCTGGTTCAAA |
| MLCK R (SEQ ID NO: 8) | GCAGGTGTACTTGGCATCGT |
| FN1 F (SEQ ID NO: 9) | AGCCGAGGTTTTAACTGCGA |
| FN1 R (SEQ ID NO: 10) | CCCACTCGGTAAGTGTTCCC |
| Col1al F (SEQ ID NO: 11) | GAGGGCCAAGACGAAGACATC |
| Col1al R (SEQ ID NO: 12) | CAGATCACGTCATCGCACAAC |
| TGFB1 F (SEQ ID NO: 13) | TACCTGAACCCGTGTTGCTCTC |
| TGFB1 R (SEQ ID NO: 14) | GTTGCTGAGGTATCGCCAGGAA |

TABLE 2
Sequences

| | |
|---|---|
| GAPDH | MAPQMYEFHLPLSPEELLKSGGVNQYVVQEVLSIKHLPPQLRA |
| | FQAAFRAQGPLAMLQHFDTIYSILHHFRSIDPGLKEDTLQFLI |
| | KVVSRHSQELPAILDDTTLSGSDRNAHLNALKMNCYALIRLLE |
| | SFETMASQTNLVDLDLGGKGKKARTKAAHGFDWEEERQPILQL |
| | LTQLLQLDIRHLWNHSIIEEEFVSLVTGCCYRLLENPTINHQK |
| | NRPTREAITHLLGVALTRYNHMLSATVKIIQMLQHFEHLAPVL |
| | VAAVSLWATDYGMKSIVGEIVREIGQKCPQELSRDPSGTKGFA |
| | AFLTELAERVPAILMSSMCILLDHLDGENYMMRNAVLAAMAEM |
| | VLQVLSGDQLEAAARDTRDQFLDTLQAHGHDVNSFVRSRVLQL |
| | FTRIVQQKALPLTRFQAVVALAVGRLADKSVLVCKNAIQLLAS |
| | FLANNPFSCKLSDADLAGPLQKETQKLQEMRAQRRTAAASAVL |
| | DPEEEWEAMLPELKSTLQQLLQLPQGEEEIPEQIANTETTEDV |
| | KGRIYQLLAKASYKKAIILTREATGHFQESEPFSHIDPEESEE |
| | TRLLNILGLIFKGPAASTQEKNPRESTGNMVTGQTVCKNKPNM |
| | SDPEESRGNDELVKQEMLVQYLQDAYSFSRKITEAIGIISKMM |
| | YENTTTVVQEVIEFFVMVFQFGVPQALFGVRRMLPLIWSKEPG |
| | VREAVLNAYRQLYLNPKGDSARAKAQALIQNLSLLLVDASVGT |
| | IQCLEEILCEFVQKDELKPAVTQLLWERATEKVACCPLERCSS |
| | VMLLGMMARGKPEIVGSNLDTLVSIGLDEKFPQDYRLAQQVCH |
| | AIANISDRRKPSLGKRHPPFRLPQEHRLFERLRETVTKGFVHP |
| | DPLWIPFKEVAVTLIYQLAEGPEVICAQILQGCAKQALEKLEE |
| | KRTSQEDPKESPAMLPTFLLMNLLSLAGDVALQQLVHLEQAVS |
| | GELCRRRVLREEQEHKTKDPKEKNTSSETTMEEELGLVGATAD |
| | DTEAELIRGICEMELLDGKQTLAAFVPLLLKVCNNPGLYSNPD |
| | LSAAASLALGKFCMISATFCDSQLRLLFTMLEKSPLPIVRSNL |
| | MVATGDLAIRFPNLVDPWTPHLYARLRDPAQQVRKTAGLVMTH |
| | LILKDMVKVKGQVSEMAVLLIDPEPQIAALAKNFFNELSHKGN |
| | AIYNLLPDIISRLSDPELGVEEEPFHTIMKQLLSYITKDKQTE |
| | SLVEKLCQRFRTSRTERQQRDLAYCVSQLPLTERGLRKMLDNF |
| | DCFGDKLSDESIFSAFLSVVGKLRRGAKPEGKAIIDEFEQKLR |
| | ACHTRGLDGIKELEIGQAGSQRAPSAKKPSTGSRYQPLASTAS |
| | DNDFVTPEPRRTTRRHPNTQQRASKKKPKVVFSSDESSEEDLS |
| | AEMTEDETPKKTTPILRASARRHRS |
| aSMA | MCDEDETTALVCDNGSGLVKAGFAGDDAPRAVFPSIVGRPRHQ |
| | GVMVGMGQKDSYVGDEAQSKRGILTLKYPIEHGIITNWDDMEK |
| | IWHHTFYNELRVAPEEHPTLLTEAPLNPKANREKMTQIMFETF |
| | NVPAMYVAIQAVLSLYASGRTTGIVLDSGDGVTHNVPIYEGYA |
| | LPHAIMRLDLAGRDLTDYLMKILTERGYSFVTTAEREIVRDIK |
| | EKLCYVALDFENEMATAASSSSLEKSYELPDGQVITIGNERFR |
| | CPETLFQPSFIGMESAGIHETTYNSIMKCDIDIRKDLYANNVM |
| | SGGTTMYPGIADRMQKEITALAPSTMKIKIIAPPERKYSVWIG |
| | GSILASLSTFQQMWITKQEYDEAGPSIVHRKCF |
| SMAD2 | MSSILPFTPPVVKRLLGWKKSAGGSGGAGGGEQNGQEEKWCEK |
| | AVKSLVKKLKKTGRLDELEKAITTQNCNTKCVTIPSTCSEIWG |
| | LSTPNTIDQWDTTGLYSFSEQTRSLDGRLQVSHRKGLPHVIYC |
| | RLWRWPDLHSHHELKAIENCEYAFNLKKDEVCVNPYHYQRVET |
| | PVLPPVLVPRHTEILTELPPLDDYTHSIPENTNFPAGIEPQSN |
| | YIPETPPPGYISEDGETSDQQLNQSMDTGSPAELSPTTLSPVN |
| | HSLDLQPVTYSEPAFWCSIAYYELNQRVGETFHASQPSLTVDG |
| | FTDPSNSERFCLGLLSNVNRNATVEMTRRHIGRGVRLYYIGGE |
| | VFAECLSDSAIFVQSPNCNQRYGWHPATVCKIPPGCNLKIFNN |
| | QEFAALLAQSVNQGFEAVYQLTRMCTIRMSFVKGWGAEYRRQT |
| | VTSTPCWIELHLNGPLQWLDKVLTQMGSPSVRCSSMS |
| MLCK | MDTKLNMLNE KVDQLLHFQE DVTEKLQSMC RDMGHLERGL |
| | HRLEASRAPG PGGADGVPHI DTQAGWPEVL ELVRAMQQDA |
| | AQHGARLEAL FRMVAAVDRA IALVGATFQK SKVADFLMQG |
| | RVPWRRGSPG DSPEENKERV EEEGGKPKHV LSTSGVQSDA |
| | REPGEESQKA DVLEGTAERL PPIRASGLGA DPAQAVVSPG |
| | QGDGVPGPAQ AFPGHLPLPT KVEAKAPETP SENLRTGLEL |
| | APAPGRVNVV SPSLEVAPGA GQGASSSRPD PEPLEEGTRL |
| | TPGPGPQCPG PPGLPAQARA THSGGETPPR ISIHIQEMDT |
| | PGEMLMTGRG SLGPTLTTEA PAAAQPGKQG PPGTGRCLQA |
| | PGTEPGEQTP EGARELSPLQ ESSSPGGVKA EEEQRAGAEP |
| | GTRPSLARSD DNDHEVGALG LQQGKSPGAG NPEPEQDCAA |
| | RAPVRAEAVR RMPPGAEAGS VVLDDSPAPP APFEHRVVSV |
| | KETSISAGYE VCQHEVLGGG RFGQVHRCTE KSTGLPLAAK |
| | IIKVKSAKDR EDVKNEINIM NQLSHVNLIQ LYDAFESKHS |
| | CTLVMEYVDG GELFDRITDE KYHLTELDVV LFTRQICEGV |
| | HYLHQHYILH LDLKPENILC VNQTGHQIKI IDFGLARRYK |
| | PREKLKVNFG TPEFLAPEVV NYEFVSFPTD MWSVGVITYM |
| | LLSGLSPFLG ETDAETMNFI VNCSWDFDAD TFEGLSEEAK |
| | DFVSRLLVKE KSCRMSATQC LKHEWLNNLP AKASRSKTRL |
| | KSQLLLQKYI AQRKWKKHFY VVTAANRLRK FPTSP |
| FN1 | MLRGPGPGLLLLAVQCLGTAVPSTGASKSKRQAQQMVQPQSPV |
| | AVSQSKPGCYDNGKHYQINQQWERTYLGNALVCTCYGGSRGFN |
| | CESKPEAEETCFDKYTGNTYRVGDTYERPKDSMIWDCTCIGAG |
| | RGRISCTIANRCHEGGQSYKIGDTWRRPHETGGYMLECVCLGN |
| | GKGEWTCKPIAEKCFDHAAGTSYVVGETWEKPYQGWMMVDCTC |
| | LGEGSGRITCTSRNRCNDQDTRTSYRIGDTWSKKDNRGNLLQC |
| | ICTGNGRGEWKCERHTSVQTTSSGSGPFTDVRAAVYQPQPHPQ |
| | PPPYGHCVTDSGVVYSVGMQWLKTQGNKQMLCTCLGNGVSCQE |
| | TAVTQTYGGNSNGEPCVLPFTYNGRTFYSCTTEGRQDGHLWCS |
| | TTSNYEQDQKYSFCTDHTVLVQTRGGNSNGALCHFPFLYNNHN |
| | YTDCTSEGRRDNMKWCGTTQNYDADQKFGFCPMAAHEEICTTN |
| | EGVMYRIGDQWDKQHDMGHMMRCTCVGNGRGEWTCIAYSQLRD |
| | QCIVDDITYNVNDTFHKRHEEGHMLNCTCFGQGRGRWKCDPVD |
| | QCQDSETGTFYQIGDSWEKYVHGVRYQCYCYGRGIGEWHCQPL |
| | QTYPSSSGPVEVFITETPSQPNSHPIQWNAPQPSHISKYILRW |
| | RPKNSVGRWKEATIPGHLNSYTIKGLKPGVVYEGQLISIQQYG |
| | HQEVTRFDFTTTSTSTPVTSNTVTGETTPFSPLVATSESVTEI |
| | TASSFVVSWVSASDTVSGFRVEYELSEEGDEPQYLDLPSTATS |
| | VNIPDLLPGRKYIVNVYQISEDGEQSLILSTSQTTAPDAPPDT |
| | TVDQVDDTSIVVRWSRPQAPITGYRIVYSPSVEGSSTELNLPE |
| | TANSVTLSDLQPGVQYNITIYAVEENQESTPVVIQQETTGTPR |
| | SDTVPSPRDLQFVEVTDVKVTIMWTPPESAVTGYRVDVIPVNL |
| | PGEHGQRLPISRNTFAEVTGLSPGVTYYFKVFAVSHGRESKPL |
| | TAQQTTKLDAPTNLQFVNETDSTVLVRWTPPRAQITGYRLTVG |
| | LTRRGQPRQYNVGPSVSKYPLRNLQPASEYTVSLVAIKGNQES |
| | PKATGVFTTLQPGSSIPPYNTEVTETTIVITWTPAPRIGFKLG |
| | VRPSQGGEAPREVTSDSGSIVVSGLTPGVEYVYTIQVLRDGQE |
| | RDAPIVNKVVTPLSPPTNLHLEANPDTGVLTVSWERSTTPDIT |
| | GYRITTTPTNGQQGNSLEEVVHADQSSCTFDNLSPGLEYNVSV |
| | YTVKDDKESVPISDTIIPEVPQLTDLSFVDITDSSIGLRWTPL |
| | NSSTIIGYRITVVAAGEGIPIFEDFVDSSVGYYTVTGLEPGID |
| | YDISVITLINGGESAPTTLTQQTAVPPPTDLRFTNIGPDTMRV |
| | TWAPPPSIDLTNFLVRYSPVKNEEDVAELSISPSDNAVVLTNL |
| | LPGTEYVVSVSSVYEQHESTPLRGRQKTGLDSPTGIDFSDITA |
| | NSFTVHWIAPRATITGYRIRHHPEHFSGRPREDRVPHSRNSIT |
| | LTNLTPGTEYVVSIVALNGREESPLLIGQQSTVSDVPRDLEVV |
| | AATPTSLLISWDAPAVTVRYYRITYGETGGNSPVQEFTVPGSK |
| | STATISGLKPGVDYTITVYAVTGRGDSPASSKPISINYRTEID |
| | KPSQMQVTDVQDNSISVKWLPSSSPVTGYRVTTTPKNGPGPTK |
| | TKTAGPDQTEMTIEGLQPTVEYVVSVYAQNPSGESQPLVQTAV |
| | TNIDRPKGLAFTDVDVDSIKIAWESPQGQVSRYRVTYSSPEDG |
| | IHELFPAPDGEEDTAELQGLRPGSEYTVSVVALHDDMESQPLI |
| | GTQSTAIPAPTDLKFTQVTPTSLSAQWTPPNVQLTGYRVRVTP |
| | KEKTGPMKEINLAPDSSSVVVSGLMVATKYEVSVYALKDTLTS |
| | RPAQGVVTTLENVSPPRRARVTDATETTITISWRTKTETITGF |
| | QVDAVPANGQTPIQRTIKPDVRSYTITGLQPGTDYKIYLYTLN |
| | DNARSSPVVIDASTAIDAPSNLRFLATTPNSLLVSWQPPRARI |
| | TGYIIKYEKPGSPPREVVPRPRPGVTEATITGLEPGTEYTIYV |
| | IALKNNQKSEPLIGRKKTDELPQLVTLPHPNLHGPEILDVPST |
| | VQKTPFVTHPGYDTGNGIQLPGTSGQQPSVGQQMIFEEHGFRR |
| | TTPPTTATPIRHRPRPYPPNVGEEIQIGHIPREDVDYHLYPHG |
| | PGLNPNASTGQEALSQTTISWAPFQDTSEYIISCHPVGTDEEP |
| | LQFRVPGTSTSATLTGLTRGATYNVIVEALKDQQRHKVREEVV |
| | TVGNSVNEGLNQPTDDSCFDPYTVSHYAVGDEWERMSESGFKL |
| | LCQCLGFGSGHFRCDSSRWCHDNGVNYKIGEKWDRQGENGQMM |
| | SCTCLGNGKGEFKCDPHEATCYDDGKTYHVGEQWQKEYLGAIC |
| | SCTCFGGQRGWRCDNCRRPGGEPSPEGTTGQSYNQYSQRYHQR |
| | TNTNVNCPIECFMPLDVQADREDSRE |
| Col1al | MFSFVDLRLLLLLAATALLTHGQEEGQVEGQDEDIPPITCVQN |
| | GLRYHDRDVWKPEPCRICVCDNGKVLCDDVICDETKNCPGAEV |
| | PEGECCPVCPDGSESPTDQETTGVEGPKGDTGPRGPRGPAGPP |
| | GRDGIPGQPGLPGPPGPPGPPGPPGLGGNFAPQLSYGYDEKST |
| | GGISVPGPMGPSGPRGLPGPPGAPGPQGFQGPPGEPGEPGASG |
| | PMGPRGPPGPPGKNGDDGEAGKPGRPGERGPPGPQGARGLPGT |
| | AGLPGMKGHRGFSGLDGAKGDAGPAGPKGEPGSPGENGAPGQM |
| | GPRGLPGERGRPGAPGPAGARGNDGATGAAGPPGPTGPAGPPG |
| | FPGAVGAKGEAGPQGPRGSEGPQGVRGEPGPPGPAGAAGPAGN |
| | PGADGQPGAKGANGAPGIAGAPGFPGARGPSGPQGPGGPPGPK |
| | GNSGEPGAPGSKGDTGAKGEPGPVGVQGPPGPAGEEGKRGARG |
| | EPGPTGLPGPPGERGGPGSRGFPGADGVAGPKGPAGERGSPGP |
| | AGPKGSPGEAGRPGEAGLPGAKGLTGSPGSPGPDGKTGPPGPA |
| | GQDGRPGPPGPPGARGQAGVMGFPGPKGAAGEPGKAGERGVPG |
| | PPGAVGPAGKDGEAGAQGPPGPAGPAGERGEQGPAGSPGFQGL |
| | PGPAGPPGEAGKPGEQGVPGDLGAPGPSGARGERGFPGERGVQ |
| | GPPGPAGPRGANGAPGNDGAKGDAGAPGAPGSQGAPGLQGMPG |
| | ERGAAGLPGPKGDRGDAGPKGADGSPGKDGVRGLTGPIGPPGP |
| | AGAPGDKGESGPSGPAGPTGARGAPGDRGEPGPPGPAGFAGPP |
| | GADGQPGAKGEPGDAGAKGDAGPPGPAGPAGPPGPIGNVGAPG |
| | AKGARGSAGPPGATGFPGAAGRVGPPGPSGNAGPPGPPGPAGK |
| | EGGKGPRGETGPAGRPGEVGPPGPPGPAGEKGSPGADGPAGAP |
| | GTPGPQGIAGQRGVVGLPGQRGERGFPGLPGPSGEPGKQGPSG |
| | ASGERGPPGPMGPPGLAGPPGESGREGAPGAEGSPGRDGSPGA |
| | KGDRGETGPAGPPGAPGAPGAPGPVGPAGKSGDRGETGPAGPA |
| | GPVGPVGARGPAGPQGPRGDKGETGEQGDRGIKGHRGFSGLQG |
| | PPGPPGSPGEQGPSGASGPAGPRGPPGSAGAPGKDGLNGLPGP |
| | IGPPGPRGRTGDAGPVGPPGPPGPPGPPGPPSAGFDFSFLPQP |
| | PQEKAHDGGRYYRADDANVVRDRDLEVDTTLKSLSQQIENIRS |
| | PEGSRKNPARTCRDLKMCHSDWKSGEYWIDPNQGCNLDAIKVF |
| | CNMETGETCVYPTQPSVAQKNWYISKNPKDKRHVWFGESMTDG |
| | FQFEYGGQGSDPADVAIQLTFLRLMSTEASQNITYHCKNSVAY |
| | MDQQTGNLKKALLLQGSNEIEIRAEGNSRFTYSVTVDGCTSHT |
| | GAWGKTVIEYKTTKTSRLPIIDVAPLDVGAPDQEFGFDVGPVC |
| | FL |
| TGFB1 | MPPSGLRLLPLLLPLLWLLVLTPGRPAAGLSTCKTIDMELVKR |
| | KRIEAIRGQILSKLRLASPPSQGEVPPGPLPEAVLALYNSTRD |
| | RVAGESAEPEPEPEADYYAKEVTRVLMVETHNEIYDKFKQSTH |
| | SIYMFFNTSELREAVPEPVLLSRAELRLLRLKLKVEQHVELYQ |
| | KYSNNSWRYLSNRLLAPSDSPEWLSFDVTGVVRQWLSRGGEIE |
| | GFRLSAHCSCDSRDNTLQVDINGFTTGRRGDLATIHGMNRPFL |
| | LLMATPLERAQHLQSSRHRRALDTNYCFSSTEKNCCVRQLYID |
| | FRKDLGWKWIHEPKGYHANFCLGPCPYIWSLDTQYSKVLALYN |
| | QHNPGASAAPCCVPQALEPLPIVYYVGRKPKVEQLSNMIVRSC |
| | KCS |

Gene expression levels were normalized to housekeeping control gene GAPDH and relative expression was computed.

Statistical Analysis was performed using the methods described in Example 2.

In proof of principle engineering studies, collagen I gels were made using normal human lung fibroblasts (HLF) grown in low serum (2% FBS) and lung adenocarcinoma (A549) cell spheroids. A549 cells were grown in F12k media with 10% FBS and when confluent cells were seeded into two columns of a Corning Elplasia 96-well plate (79 spheroids per well) at a total density of 7,500 cells/well. Spheroids were cultured for 5-7 days before being harvested and seeded into gels. A 500 uL collagen I (2.5 mg/mL) master mix was made with 350 k HLF/ml and ˜632 spheroids (16 wells), which was then plated on a 6 well plate at 50 uL per gel droplet. After 24 hours, gel droplets were detached from the plate and allowed to contract freely for 7 days establishing our high-density tumor stroma tissues. Allowing the cells to contract the collagen is the key step to creating a dense construct that far exceeds the original collagen concentration. In this study, control gels contained HLF only—for the purposes of demonstrating that incorporating cancer spheroids creates heterogeneous patterns of ECM. Gels were fixed using 4% PFA and were then paraffin embedded to obtain slices of the gels. For end-point analysis, we conducted immunohistochemistry staining for extracellular matrix markers including collagen I and fibronectin, confocal microscopy, and various image analysis metrics provided by the FIJI macro TWOMBLI to quantify the percentage of collagen density.

### Results and Discussion

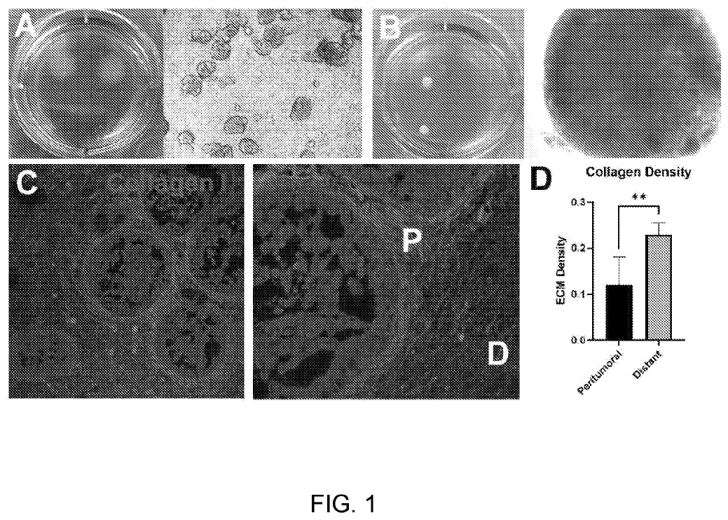

Tumor constructs contracted continuously over the 7-day period (FIGS. 1A and 1B). Our estimates of the change in tissue volume based on displacement measurements in a graduated microfuge tube suggest that the collagen concentration increases to greater than 100 mg/ml. Collagen density in peritumoral regions was ˜2 times greater than at distant locations. Reorganization of collagen fibers to a concentric pattern enrobing the tumor islands recapitulated observe patterns of collagen at solid tumor fronts in vivo (peritumoral, FIG. 1C). Collagen matrix reorganization during tissue contraction was accompanied by synthesize of a dense fibronectin matrix that localized most intensely at tumor island interfaces. Regarding future applications, research has shown that tumor cell invasion by T cells through tissue is highly regulated and limited by properties of the stromal ECM, including collagen density and concentric fiber orientation. Our approach to engineering 3D cancer models with a dense stroma may enable realistic testing of T cell immunotherapy in vitro.

### Example 2. Cellular Sculpting of Collagen Hydrogels as a Tool for Engineering 3D Models of Dense Solid Tumors with Infiltrating Stroma

The structural features and mechanical properties of solid tumors influence cancer progression and treatment response. While it has long been possible to engineer culture substrates and scaffolds with stiffnesses that approximate tumors, these models often fail to capture the structural features of spatial confinement and packing that occur in cancers with dense infiltrating stroma. For example, patterns of densely compacted concentric ECM fibers often seen at the stromal interface in carcinomas are believed to restrict the access of molecules and cells to the tumor cells. Contractile cells can solve this engineering problem by virtue of their ability to contract and deform extracellular matrix (ECM) fibers, which leads to the progressive removal of interstitial fluid if the construct is allowed to freely contract. Here we demonstrate that allowing human fibroblasts to freely contract type I hydrogels co-seeded with carcinoma cell spheroids produces a tissue engineered construct with structural features that mimic stroma-rich invasive carcinomas in vivo. We used a combination of histological, morphometric, mechanical, and genetic analyses to confirm recapitulation of microarchitectural, mechanical, and molecular features of solid tumors with a dense infiltrating stroma. Our study establishes a paradigm for engineering 3D cancer models with dense stroma that offers user-defined control of the initial cellular and ECM inputs. The physical characteristics of the dense carcinoma constructs engineered using this approach provide a previously nonexistent venue for studying the challenges associated with the delivery of macromolecular drugs and cellular immunotherapies to solid tumors.

### Introduction

Early work by Mina Bissell's group and others in the 1980s catalyzed a four-decade endeavor to create three-dimensional (3D) models of cancer for in vitro laboratory investigation (refs). From a tissue engineering standpoint, the creation of 3D cancer models that capture the physical continuum of ECM density, spatial confinement, bulk mechanical properties, and heterogeneous patterns of structural anisotropy present in solid tumors remains a challenge. In vitro studies using 3D cultures confirmed the hypothesis that increased tension in the TME drives malignant phenotypes (Weaver papers). Experiments using single cancer cells and small aggregates with parallel multiscale continuum model-based simulation revealed that local stiffening and anisotropic rearrangement of the ECM drove tumor cell invasion in low-density collagen type I hydrogels (Shenoy papers). There are numerous hydrogel systems with tunable elastic moduli which have been used to study the effect of tissue stiffness on cellular processes such as migration and lineage specification in progenitor cells (Discher and others); however, the interrelationship of altered bulk mechanical properties and heterogeneous structural features such as local anisotropy remains unclear. Furthermore, synthetic hydrogels stiffened via increasing the concentration of polymer or the degree of cross-linking fail to create a commensurate change in fiber density, require chemical modification to enable cell adhesion, and are often designed for pseudo-3D surface cultures on which adhesion occurs primarily in a single plane (Discher etc). Hydrogels used for in vitro organoid and explant cultures such as Matrigel and collagen type I gels at commercially available concentrations fail to capture the mechanics of spatial confinement and packing of tumor cells that occurs in solid tumors.

The combination of mechanical and structural mimicry is necessary for accurate modeling of cancer processes relevant to treatment resistance. Drug resistance in cancers has traditionally been attributed to resistant tumor cell phenotypes, which may be intrinsic to driver mutations or may evolve via further mutation and selection pressures imposed by cytotoxic therapies (refs). This conceptual model omits the role of physical constraints imposed on therapy by regions of dense and relatively avascular stroma in solid tumors, including longer diffusion distances, more tortuous routes of diffusion in the interstitium, and increased interstitial fluid pressure driving interstitial flow away from the tumor core (refs). Similarly, the translation of promising cellular immunotherapies for the treatment of solid tumors is hampered by the inability of sufficiently large cell numbers to traverse the complex physical barriers in a dense and compacted TME (refs). These observations highlight the importance of considering the structural features of solid tumors in the engineering design of 3D cancer models.

Tissue-engineered cancer models that capture challenges to effective therapy imposed by physical constraints of a solid tumor will facilitate and accelerate the pace of front-end hypothesis generating investigations and enhance the accuracy of therapeutic screening studies. Our goal is to recapitulate these heterogeneous structural and mechanical patterns in a tissue engineered system. From a tissue engineering standpoint, first principles dictate that a minimal form of such a model must include a fraction of tumor mass, a fraction of dense collagenous ECM, and a fraction of fibroblasts which are the primary synthesizers of collagen and generators of tension in the ECM via application of contractile force generated by cytoskeletal actomyosin machinery. Cancer-associated fibroblasts (CAF) play a key role in the formation of a dense stroma via excess ECM synthesis, thereby mirroring the roles of activated myofibroblasts in tissue fibrosis. Standard primary fibroblast cultures fibroblast cultures with one or more points of tension around a synthetic element or post aside from the simple tension caused by ECM and fibroblasts wrapped around the cancer cells can create an artificial amount of compaction that likely disrupts the most reliable cancer models from being formulated.

### Methods

### Cell Culture

Normal human lung fibroblasts (HLFs) [American Type Culture Collection (ATCC) and Lonza] were cultured in fibroblast basal medium with low serum kit (ATCC) and 2% fetal bovine serum (FBS). A549 lung adenocarcinoma cells (ATCC) were cultured in F-12K medium supplemented with 10% FBS. HT-29 colorectal adenocarcinoma cells (ATCC) were cultured in McCoy's 5A supplemented with 10% FBS. MCF-7 breast adenocarcinoma cells were cultured in DMEM or F12K with 10% FBS. All culture media contained 1% antibiotic-antimycotic (Corning) and cultured in a humidified tissue culture incubator at 37° C. with 5% CO2. Typical passage numbers used. Confluency at splits.

### Constructing Dense Carcinoma Tissues

Cells were harvested and collected using standard trypsinization. A 2.5 mg/ml collagen I hydrogel solution was prepared according to manufacturer protocols (Corning). HLF were introduced into the collagen mixture at a density of 2.5-5.0×10⁵ cells/ml and gel droplets were made by pipetting 50 uL of gel solution onto a 6 well plate. Higher initial cell densities produced more rapid contraction which was not desired for the current study. Dense carcinoma constructs were formed by co-seeding carcinoma cell spheroids (A549, MCF7, or HT29) along with HLF. Carcinoma spheroids were cultured for 5 days in low attachment plates (Corning) prior to tissue construct formation. Constructs were cultured for cultured in the plate-bound format for 24 hours in FGM with 2% FBS. After 24 hours, constructs were manually detached from plate surfaces using a pipet tip or sterilized spatula and allowed to freely contract for 1, 4, or 7 days in the same culture medium prior to collection for end-point analysis.

### Estimating Collagen Density

We assumed an ellipsoidal geometry of constructs with a proportional circular base diameter and by measuring the depth of the gel using the top-down images, we estimated the volumes of the construct at the various time points. The final volume gives a rough estimate of the gel volume ellipsoid geometry. Estimated final volumes were used to calculate an estimated new collagen concentration after contraction by assuming that the initial mass of collagen is conserved during contraction and ignoring any cell-elaborated collagen type I.

### Immunohistochemistry

Paraformaldehyde-fixed tissues were taken to the Pathology Core Laboratory at Tulane University Health Sciences Center for paraffin embedding and sectioning. Hematoxylin and eosin (H&E) stained sections were provided by the core. Paraffin sections were deparaffinized, rehydrated and processed for immunohistochemical staining using standard procedures. After rehydration, sections were washed with PBS, blocked and permeabilized in PBS containing 3% bovine serum albumin (BSA) and 0.1% Triton-X, prepared in PBS for at least 30 min at room temperature, then incubated for 2-3 hours at room temperature with primary antibodies against collagen type I (Abcam, ab34710)) or fibronectin (Abcam, abXYZ) at dilutions of 1:200 in PBS with 1% BSA. After washing, secondary antibodies were incubated at dilution of 1:1000 along with Hoescht dye as a nuclear counterstain in PBS with 1% BSA for 30 minutes at room temperature. After final washes in PBS, slides were mounted with antifade medium (Vector Labs) and stored prior to fluorescence microscopy.

### Microscopy and Image Analysis

All slides were imaged on a Nikon Ti-2 Confocal Microscope and images were acquired using a Nikon DS-FI3 camera. Collagen density and directional orientation were quantified via analysis of fluorescence micrographs. Images for quantitative analysis were acquired with the same exposure times and laser power settings. For collagen density analysis, fluorescence intensity was measured in FIJI across 30 μm in four regions of interest, starting at a tangent line to the spheroid interface and moving outward along a perpendicular line. ROIs were 50×10 μm with the longer side of each ROI running parallel to the initial tangent of the spheroid. At least 10 sections were imaged analyzed for each experimental group and time point. Collagen directionality analysis was performed using micrographs of H&E stained slides and the FIJI directionality plugin. Orientation of tissue structures labeled by eosin was measured in ROIs extending 45 μm from the spheroid interface. Orientation refers to degrees from the tangent line. Percent goodness of fit of a Gaussian curve to the orientation histogram produced in FIJI was used to assess variance in the data for each ROI. At least 10 sections were imaged analyzed for each experimental group and time point.

### Mechanical Testing

A nanotribometer (CETR, UMT Multispecimen Test System) with the force sensitivity of 1 μN [10] was used to investigate the stress-strain response of the soft gels under ambient conditions. In a typical run, a gel sample (typically thickness of 0.8-1.2 mm) was placed on a glass slide. Phosphate buffered saline was added to the gel sample to not only preserve the hydration and structural integrity of the tissue construct, but also to minimize the adhesive interaction between the probe and the substrate. A stainless steel cylindrical-shaped rod with a diameter of 1 mm was used as the probe with an approach velocity of 0.065 mm/s. A cylindrical-shaped probe was chosen to maintain a constant contact area with the sample's surface thereby minimizing errors associated with adhesive dissipation energy [11]. In all experiments, surface contact and indentation depth were controlled to obtain reproducible test results [12]. Effects of vibrations from the test systems were also dampened using aluminum foil coating to prevent fluctuations in measurements. The initial contact was determined by locating the force increase in the force vs time data. Then the initial linear region of this force vs time data was used to calculate a stress and strain values:

$$σ ( t ) = F z ( t ) / Ap \qquad ( 1 )$$

$$ϵ ( t ) = z ( t ) - z 0 - Fz ( t ) / k * th \qquad ( 2 )$$

where σ(t) is the stress at time t, Fz(t) is the force applied to the gel at time t, Ap is the surface area of the probe, ε(t) is the strain at time t, z(t) is the height of the probe at time t, z0 is the height of the probe when it makes contact with the gel, k is the spring constant, and th is the thickness of the gel. Thickness of the gel was measured in Fiji via micrographs of each gel. The resulting stress/strain curve was used to obtain the linear response of material to the compressive stress. A linear regression line was then fit to the curve. The slope of this regression line was equal to the elastic modulus (E) of the gel sample. Six total groups of 50 ml gels were tested (500K HLFs/ml and 0 HLFs/ml for day 1,4, and 7) with six replicates per group. Day 1 refers to the day at which gels were detached from the well plate. Results of the three closest elastic moduli values were selected as representative replicates from each group. All data analysis was performed in R.

### Gene Expression Analysis

Constrained and unconstrained were harvested after 7 days of culture and stabilized in RNALater tissue reagent prior to RNA isolation. Isolation of total RNA from gels was adapted from Qiagen RNeasy manufacturer's protocol (Qiagen GmBH, Hilden, Germany), and RNA purity was measured. For cDNA synthesis, 1 μg of RNA was used with qScript cDNA supermix (Quantabio) according to manufacturer's instructions. The final cDNA sample was diluted in a 1:10 ratio in ultrapure water and stored in −20° C. until use. RT-qPCR occurred using Thermofischer Step One Plus instrument and was quantified using SYBR Green Power Up master mix. Gene expression levels were normalized to housekeeping control gene GAPDH and relative expression was computed.

The following primer sequences were used:

GAPDH F
(TTAAAAGCAGCCCTGGTGAC),
GAPDH R
(CTCTGCTCCTCCTGTTCGAC)
aSMA F
(CCGACCGAATGCAGAAGGA),
aSMA R
(ACAGAGTATTTGCGCTCCGAA)
SMAD2 F
(ATGTCGTCCATCTTGCCATTC),
SMAD2 R
(AACCGTCCTGTTTTCTTTAGCTT)
MLCK F
(CCCGAGGTTGTCTGGTTCAAA),
MLCK R
(GCAGGTGTACTTGGCATCGT)
FN1 F
(AGCCGAGGTTTTAACTGCGA),
FN1 R
(CCCACTCGGTAAGTGTTCCC)
Col1al F
(GAGGGCCAAGACGAAGACATC),
Col1al R
(CAGATCACGTCATCGCACAAC)
TGFB1 F
(TACCTGAACCCGTGTTGCTCTC),
TGFB1 R
(GTTGCTGAGGTATCGCCAGGAA)

### Statistical Analysis

Data were compared using either paired or unpaired Student's t-tests, depending on experimental design. For unpaired data, when standard deviation of values in two groups was significantly different (p<0.05), Welch's unpaired Student's t-test was used. All statistical analyses were performed using GraphPad software. At p<0.05, differences were considered statistically significant.

### Results

### Cellular Sculpting of Dense Carcinoma Constructs with Prominent Stroma

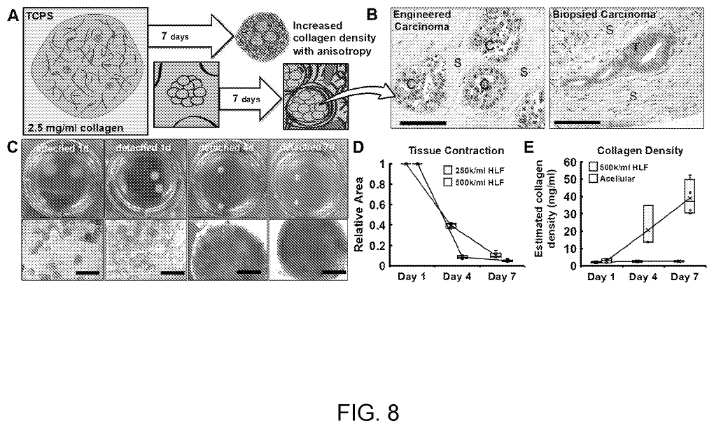

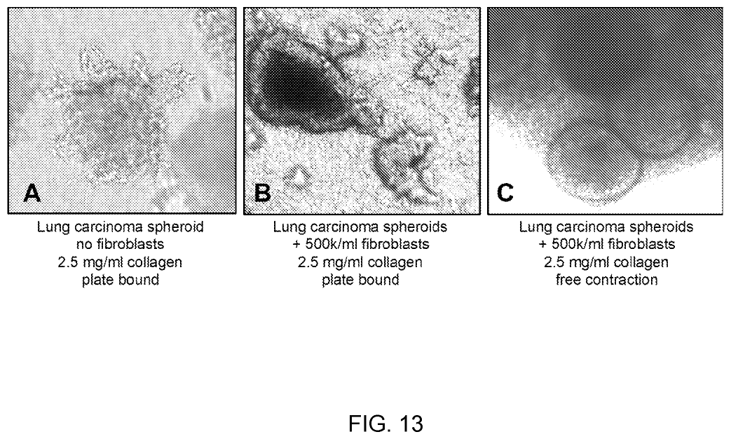

We confirmed uniform fibroblast spreading in the collagen gel architecture after overnight culture with the construct still attached to a tissue culture plate. This quality control test verifies strong adhesion and tension loading of the ECM prior to release from the plate. Continuous contraction of the released constructs was grossly observable during the 7-day culture period (FIG. 8C). We formed carcinoma constructs using HLF seeded in combination with spheroids composed of A549 lung carcinoma cells, MCF7 breast carcinoma cells, or HT29 colon carcinoma cells. We did not observe any effects of carcinoma spheroid type on the gross contraction of the constructs. Contraction for 7 days produced dense carcinoma constructs with prominent stromal fraction that mimics the histological appearance of biopsied stroma-rich carcinomas (FIG. 8B). We estimated collagen concentrations greater than 40 mg/ml based on the decreased volume of dense carcinoma constructs after 7 days of contraction (FIG. 8D, 8E). Spatial confinement and packing that occurs in the dense carcinoma constructs limits invasion and maintains a discernible tumor front (FIG. 8B, 9A). Carcinoma spheroids in constrained tissues which maintain a sparse collagen matrix readily invade the surrounding spaces with individual cells and strands of cells separating from the original spheroid (FIG. 13).

### Evolution of ECM Microarchitecture During Carcinoma Tissue Contraction

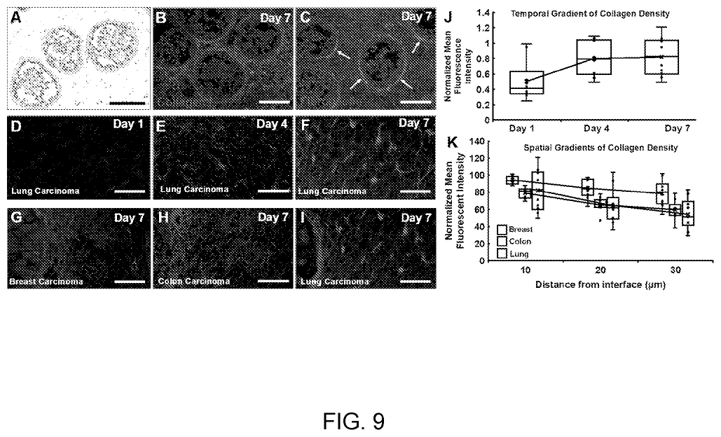

The internal microarchitecture of dense carcinoma constructs after 7 days of contraction was reminiscent of aggressive carcinomas in which tumor islands are seen embedded within a pronounced stromal fraction (FIG. 9B, 9A). Collagen type I staining of sectioned tumor constructs revealed markedly increased collagen densities surrounding tumor spheroids (FIG. 9B). Entrapment and lassoing of tumor spheroids (FIG. 9B, carcinoma cells) during the contraction process produced patterns of locally anisotropic collagen fiber orientations defined by densely packed concentric fibers seen at the interface between tumor spheroids and the surrounding stromal tissue (FIG. 9B, arrow). We stained for fibronectin in serial sections to visualize cell-elaborated ECM. We observed focal deposition at spheroid-stroma interfaces in the same interfacial regions occupied by densely packed concentric collagen fibers (FIG. 9C, arrows).

Collagen type I staining of sectioned carcinoma constructs revealed a markedly increased collagen density of during the 7 days of carcinoma construct contraction, as inferred by the fluorescence intensity of collagen type I staining (FIG. 9D, 9E, 9F). We measured the mean fluorescence intensity in these cohorts of micrographs to quantify a temporal gradient of increasing collagen content (FIG. 9J). We used the same staining and image analysis method to measure the fluorescence intensity at discrete distances from the interface of a carcinoma spheroid and the surrounding stromal tissue (FIG. 9G, 9H, 9I). We quantified a gradient of decreasing fluorescence intensity with increased distance from the interface for each type of carcinoma construct (FIG. 9K). These data demonstrate proof-of-concept for engineering carcinoma constructs with a prominent and dense stroma that contain gradients of increasing ECM density at the stromal interface with carcinoma cells, a key feature of ECM structural heterogeneity observed in stroma-rich carcinomas in vivo.

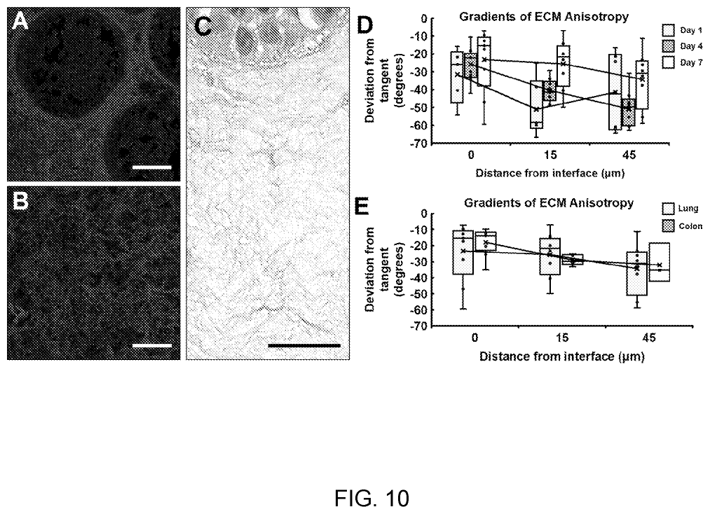

Anisotropic structure of the ECM is another defining characteristic of tumor stroma with differing hypotheses regarding the effects on tumor progression, depending on the orientation of anisotropic features relative to the direction of tumor invasion (19). ECM aligned perpendicularly to the tumor front is believed to promote tumor cell invasion via directional migratory cues (20). Conversely, ECM aligned parallel to the tumor front (concentric wrapping) is believed to restrict cell movement and promote dense packing as the tumor mass expands (21). We qualitatively observed patterns of concentric collagen orientation in the densely-packed regions adjacent to carcinoma spheroids (FIG. 10A). Constructs containing HLF only contract to a similar final volume but exhibit a uniformly isotropic microarchitecture (FIG. 10B). We used H&E-stained sections to quantify the orientation of tissue structures at discrete locations relative to the interface with carcinoma spheroids (FIG. 10C). We used deviation from a tangent line at the spheroid front as a metric to quantify parallel orientation of fibers that is indicative of concentric wrapping during the contraction process (FIG. 10D, 10E). Our analysis revealed a gradient of anisotropy at the spheroid-stroma interface. The parallel arrangement of fibers at the interface degrades gradually within a 50 □m of the interface (FIG. 10D). Our analysis revealed that anisotropy expands to greater distances with extended time of contraction (Day 1, Day, Day 7 in FIG. 10D). This trend suggests that the processes of lassoing and packing which give rise to local anisotropy at the interface continue in a progressive manner until an equilibrium volume is reached after the period of contraction. There were no significant effects of the carcinoma spheroid type on measured patterns of anisotropy (FIG. 10E). We calculated the fit goodness for the orientation analysis as a means of gauging the consistency of orientation result at each discrete location.

### Fibroblast-Mediated Contraction Drives Progressive Tissue Stiffening

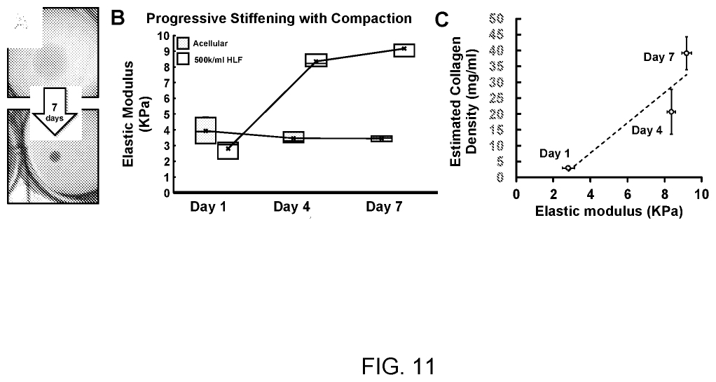

We used a nanotribometer to perform indentation testing for quantification of the clastic modulus with the instrument calibrated to soft materials of known stiffness. Tissue stiffness scales with the collagen type I content of tissues (22). Therefore, we expected a significant increase in the elastic modulus upon contraction of the tissue due to a commensurate increase in collagen type I density. We tested the hypothesis that fully contracted tissues would reach an in vivo-like density and exhibit measurable elastic moduli in the range of fibrotic tissues, approaching or greater than 10 kPa. We measured the elastic modulus at days 1, 4, and 7 to correlate tissue stiffness with macroscale contraction (FIG. 11A). The mean clastic modulus of HLF-seeded constructs was 2.8 kPa at Day 1 and increased to 8.4 kPa and 9.2 kPa at Day 4 and Day 7, respectively (FIG. 11B). The mean elastic modulus of acellular collagen gels was 3.9 KPa and 3.4 kPa after 1 and 7 days of incubation in cell culture medium, respectively. Our analysis revealed an engineerable relationship between the contraction-mediated collagen density and the measured clastic modulus (FIG. 11C).

### Gene Expression Patterns in Contracted Dense Carcinoma Tissues

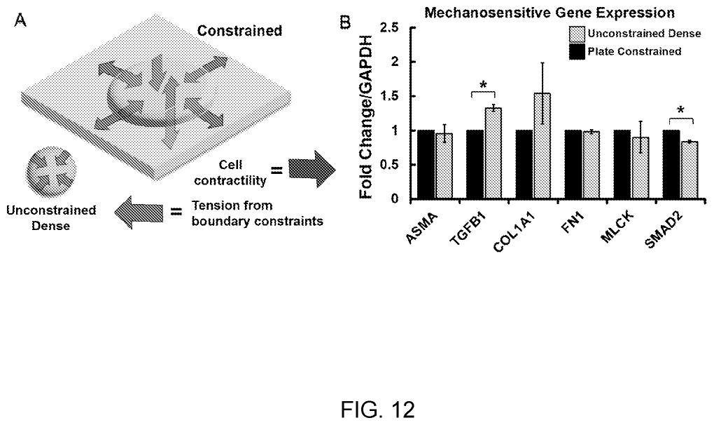

We hypothesized that the lack of externally applied tension in the absence of boundary constraints may have the unintended effect of downregulating tension-activated genes, which are characteristic drivers of stromal reactions in cancer, and tissue fibrosis more broadly (23). We compared the expression of mechanosensitive genes associated with myofibroblast differentiation and tissue fibrosis following in contracted constructs after 7 days with plate-bound constructs of the same initial composition (FIG. 12A). All genes assayed were expressed at similar levels in both groups (FIG. 12B). We measured a trend of collagen type I upregulation in the dense contracted tissues. but fibronectin expression did not change. We measured a slight but significant upregulation of TGF-B and similar downregulation of the TGF transducer SMAD2 in the dense constructs. Contractility-associated genes smooth muscle actin (ASMA) and myosin light chain kinase 2 (MLCK) were slightly downregulated in the unconstrained dense constructs. These data confirm that cells in fully contracted dense carcinoma constructs and cells in the constrained constructs express similar levels of tension-associated genes.

### Discussion

We successfully engineered 3D carcinoma tissues with a dense stromal fraction that recapitulate known histological, microarchitectural, and mechanical features of solid organ carcinomas. Key features which are not present in commonly used hydrogel-based cancer models include the dynamic transition to an in vivo-like density (FIG. 1), the temporal and spatial patterns of emergent ECM density and anisotropy (FIGS. 9 and 10), and mechanical properties in the range of biopsied solid organ carcinomas (FIG. 11). Robust invasive behavior of cancer cells, spheroids, organoids, etc., in the low protein content hydrogels used for most in vitro studies investigations occur in the absence of the physical constraints imposed on tumor growth in solid organs. We observe rapid and robust carcinoma invasion in constructs formed with typical collagen concentrations in the 2.5-5 mg/ml range, but the same carcinoma spheroids remained constrained in the dense constructs (FIG. 13). Dense carcinoma constructs exhibited a histological appearance consistent with stroma-rich carcinoma in vivo, specifically the spatial confinement of glandular structures formed by tumor cells and distinct compartmentalization from the stromal fraction (FIG. 8B). Engineering this structural feature is the critical first step toward our long-term goal of modeling the challenges of molecular and cellular access to the carcinoma cells in solid tumors with dense stroma.

We did not perform direct measurements of molecular diffusion in dense carcinoma constructs for the current study. Previous studies established that the permeability of collagen hydrogels decreases at higher collagen densities (24). We were not able to label the internal structural proteins using in-house whole mount immunohistochemistry protocols routinely used to stain lower density plate-bound constructs, due to a lack of IgG penetrance in the dense carcinoma constructs. Regarding cellular transit, we observe that carcinoma cells comprising spheroids do not appreciably invade the surrounding tissue in the dense constructs, whereas the same carcinoma cells readily invade plate-bound gels (FIG. 13). The constrained spatial environment of dense tumors influences immune cell trafficking and is therefore an important factor in the delivery of cellular immunotherapies. CAR T cell therapies are among the most promising potentially curative cancer therapies with demonstrated efficacy for hematologic malignancies, but broad efficacy in solid tumors has yet to be demonstrated (25). Immunosuppressive features of the TME are known to impeded T cell trafficking and activation in solid tumors, but it is generally accepted that the dense fibrotic ECM of the tumor stroma limits T cell penetrance into the core of solid tumors (26). In vitro studies using lung tumor explants seeded with cytotoxic T cells revealed that densely compacted patterns of concentric collagen fibers impeded invasion of the tumor cells islands (27). The dense carcinoma constructs described herein provide an ideal platform for future investigation of approaches to improve T cell penetrance in solid tumors.

The cellular sculpting process drives compaction and increasing collagen density via the expulsion of water from the bulk tissue construct. There are numerous biomaterial engineering methods for fabricating scaffolds and matrices with dense layers of collagen. Electrospinning is a versatile method that enables fabrication of dense fibrous mats using ECM proteins such as collagens and elastin (28). Dense collagen scaffolds for skin repair applications were engineered for increased stability by removing water from acellular collagen hydrogels via weighted compression on porous membranes and layers of filter paper (29). Vitrification processes that entail cyclic dehydration and rehydration of ECM hydrogels were used to create scaffolds for ocular repair and ECM-derived membranes for organ chip devices and microphysiological systems (30, 31). These versatile methods are ideal for engineering planar tissues but lack the capacity to entrap user-defined mixtures of cells and organoids in a dense 3D structure. The cellular sculpting method solves this challenge while generating spatially heterogeneous anisotropy in 3D via internal cellular contraction of the matrix fibers. The current method uses 2% FBS to promote fibroblast contractility but varying the serum concentration or adding modulators of mesenchymal cell contractility are potential approaches to engineer the rate and magnitude of contraction.

We used normal human lung fibroblasts as the contractile stromal cells for these studies, but any sufficiently contractile cells of mesenchymal origin can produce similar results. Many previous studies using a boundary anchored anisotropic tissues have demonstrated that other stromal cell types such as bone marrow-derived mesenchymal stem cells, skeletal myoblasts, and cardiomyocytes can contract collagen type I hydrogels in a commensurate fashion (18, 32-34). The starting ECM hydrogel composition may be an important and engineerable parameter for certain applications. Matrigel was admixed with collagen type I (10% v/v) in previous experiments using skeletal myoblasts to sculpt dense anisotropic tissues (Mondrinos 2021). Prior work using an array of cell types embedded in collagen type I hydrogels demonstrated that dense cell-elaborated matrices containing fibronectin, tenascin-C, and laminins are deposited within the collagen type I framework containing various mixtures of organoids and dispersed fibroblasts (Cukierman papers). Therefore, the starting ECM composition can be engineered as a means of controlling cell behaviors, or kept as monolithic collagen type I, which allows for efficient discrimination and quantification of cell-elaborated matrices as a function of defined cell compositions and culture parameters.

Reported values for the mechanical properties of collagen hydrogels vary widely, likely due to the differences in sample size and orientation, the instruments used, and the conditions of testing performed (TEB review). For example, Cross et al. measured an elastic modulus of 1.8 kPa for 2 mg/ml acellular collagen gels in compression up to 30% strain, Kobayahi et al. measured 1.6 kPa for 3 mg/ml and 2.2 kPa for 5 mg/ml, both at 10% strain, and Achilli and Mantovani measured values above 10 kPa for 2.8 mg/ml collagen gels in the 15-30% strain range (35). Strain stiffening of collagen fibers can explain the higher relative values measured at 30% vs. 10% strain. Our measured clastic modulus of 3.9 kPa for 2.5 mg/ml acellular collagen hydrogels by nanotribometry falls within the range of reported values (FIG. 11B). The lower elastic modulus of HLF-laden constructs at Day 1 was unexpected, since colloidal packing of cells within the viscous milieu of the collagen fiber network should increase mechanical strength. We hypothesize that the higher Day 1 Young's modulus measured for acellular collagen hydrogels is due to the loss of water in boundary layers of the gel during open air handling and compression on the nanotribometer stage to a greater degree than the more congealed cell-laden constructs. Dehydration in boundary layers can cause unpredictable local increases in collagen density that will increase stiffness and it is difficult to control for differences in water loss between experimental groups.

Our mechanical testing demonstrated rapid increase in the stiffness of dense tissues engineered using the cellular sculpting approach, with a 3-fold increase from 2.8 to 8.4 kPa after 4 days of cell-mediated compaction and further increase to 9.2 kPa by 7 days of culture. A study of more than 100 colon carcinomas reported an average Young's modulus of approximately 8 kPa for all tumors tested, with values of 9-13 kPa for stage III and IV tumors (36). A study of normal and pathological breast tissues found that the Young's modulus of normal adipose and glandular tissues in the breast were approximately 3.5 kPa, while low-grade invasive ductal carcinomas (IDC) had stiffened to approximately 10.5 kPa, and medium-and high-grade IDC further stiffened to the 20-40 kPa range (37). Collectively, the literature indicates that the Young's modulus of our dense constructs falls within the measured range of early stage colon and breast carcinomas, while higher grade tumors are significantly stiffer. The goal of our current study was to demonstrate that structural and mechanical properties reflective of solid organ carcinomas can be obtained with a simple 7-day culture protocol using widely available cell culture supplies. Previous studies of free-floating collagen gel contraction by fibroblasts for up to 3 weeks in culture obtained estimated collagen densities greater than 100 mg/ml using, albeit with a lower seeding density of 50,000 fibroblasts/ml vs. 250-500,000 fibroblasts/ml plus the volume fraction of carcinoma spheroids for the constructs described herein (38). We hypothesize that longer-term cultures allowing for ongoing volume reduction, deposition of endogenous ECM to increase overall density, and anisotropic rearrangement of the ECM will all further increase stiffness beyond the values obtained in the current study.

Gene expression analysis of dense carcinoma constructs suggests that the fully contracted and unconstrained dense carcinoma constructs are in a state of similar or greater mechanical activation compared to constrained constructs bound to a stiff plastic substrate (FIG. 12). Data from microphysiological models of musculoskeletal tissues used for multiscale continuum modeling revealed that patterns of structural anisotropy dramatically amplify cell contractility and reciprocal tissue stiffening along the principal axis of tension imposed by boundary constraints (18). We speculated that the unconstrained format used in this approach may result in decreased expression of tension-associated genes. It is well-established that culture on stiff and non-deformable matrices amplifies expression of tension-driven genes associated with wound healing, tissue fibrosis, and stromal reactions in cancer (39, 40). Tension is expected to be low during the early stages of contraction when the ECM deforms readily, but as the construct approaches an equilibrium volume the contractile forces exerted by the cells must be balanced by resistance of tension in the ECM. Furthermore, mechanical activation of cells occurs via parallel mechanisms including compression and packing (41). Dense carcinoma constructs engineered using the approached described herein offer a unique platform for studying the interplay of tension, compression, and packing of cells in the mechanobiology of cancers and other tissues.

### CONCLUSIONS

The current form of our dense carcinoma model demonstrates proof-of-concept for new tissue engineering models that capture the physical characteristics of cancers with prominent infiltrating stroma. Broad applicability of the current method is based on the usage of common tissue culture supplies accessible to virtually any cancer research laboratory. There are many limitations to our current model which require design refinements and subsequent engineering reduction to practice. The initial geometry of the constructs needs to be standardized to generate a more reproducible final shape of the contracted tissues. The dome shape of constructs initially cast on a plate using the current method causes occasional invagination of the flattened lower surface upon release and contraction. While the simplicity of using only cells, spheroids, collagen gel, and tissue culture plates is a strength of the current approach, there are many applications that would benefit from integrating the dense carcinoma constructs in a format that grants standardizable orientation and compartmentalized access. Future studies will focus on integrating dense carcinoma constructs within a vascularized margin tissue situated in a fluidic culture device. Our long-term goal is to create patient-derived microphysiological models of lung carcinomas and breast carcinomas to study the challenges of treatment resistance, macromolecular drug delivery, and therapeutic immune cell delivery to solid tumors with dense and prominent stroma.

1. A composition comprising:
(a) a first layer of cells comprising a plurality of cancer cells;
(b) a second layer of cells comprising a plurality of stromal cells and extracellular matrix protein; wherein:
(i) the density of the extracellular matrix protein is from about 60 to about 120 mg/mL, wherein millgrams of protein in respect to volume of cells or vessel; or
(ii) the bulk elastic modulus across the first and second layer of cells is from about 6 KPa to about 10 KPa.

2. The composition of claim 1 further comprising a hydrogel that defines a vessel within which the plurality of cancer cells and the plurality of fibroblasts are positioned, and wherein the hydrogel is absorbed or immobilized to a solid support.

3. (canceled)

4. The composition of claim 1, wherein the plurality of cancer cells are positioned in a spheroid.

5. The composition of claim 1, wherein the plurality of cancer cells comprise carcinoma cells.

6. The composition of claim 1, wherein the extracellular matrix is positioned around the plurality of cancer cells at a density of from about 7 KPa to about 9 KPa.

7. The composition of claim 1, wherein the stromal cells are at a density from about 400,000 cells per mL of volume to about 1,550,000 cell per mL of volume.

8. The composition of claim 1, wherein the plurality of cancer cells are from breast cancer carcinoma cells, skin cancer carcinoma cells or prostate cancer carcinoma cells; and/or wherein the plurality of stromal cells comprise fibroblasts.

9. The composition of claim 1, wherein the plurality of fibroblasts are free of one or more contacts points that exert tension on the plurality of cancer cells.

10. The composition of claim 1, wherein tension exerted on the plurality of cancer cells by the stromal cells is not exerted on the stromal cells by contact of the plurality of stromal cells to a point of tension other than the cancer cells.

11.-20. (canceled)

21. The composition of claim 1, wherein the second layer has anisotropic orientation around the first layer of cells.

22. (canceled)

23. The composition of claim 1, wherein the mass to mass ratio of water content in the first and second layers relative to the total mass of cells and extracellular protein is from about 65% to about 75%.

24. The composition of claim 1, wherein the plurality of stromal cells are free of one or more contacts points that exert tension on the second layer cells other than contact with the first layer of cells.

25-27. (canceled)

28. A method of assaying the toxicity or therapeutic effectiveness of an agent on a cancer cell comprising:
(a) contacting the composition of claim 1 with an agent.

29. The method of claim 28 further comprising a step of (b) monitoring the cells for morphologic changes or changes of expression profile of cells after step (a).

30. The method of claim 28, wherein the agent is chosen from one or a combination of: an environmental agent, a small molecule therapeutic, a biologic immunotherapy, or a modified T cell.

31. The method of claim 30, wherein the agent is a biologic immunotherapy that is an antibody or antibody fragment thereof.

32. The method of claim 30, wherein the agent is a modified cell that is a CAR-T cell.

33. (canceled)

34. A method of inducing mechanically activated gene expression in a cell comprising culturing the composition of claim 1 with a tissue culture media.

35. The method of claim 34, wherein the cells are in culture for at least seven days.

36. The method of claim 34 further comprising exposing the composition claim 1 to changes of one or a combination of: (i) extracellular protein density; (ii) tension; (iii) compression; and (iv) packing of cells.

37.-41. (canceled)


SEQUENCE LISTING (21 sequences)

SEQ ID NO: 1 - DNA, 20 bp, synthetic construct
  source 1..20: mol_type = other DNA
ttaaaagcagccctggtgac

SEQ ID NO: 2 - DNA, 20 bp, synthetic construct
  source 1..20: mol_type = other DNA
ctctgctcctcctgttcgac

SEQ ID NO: 3 - DNA, 19 bp, synthetic construct
  source 1..19: mol_type = other DNA
ccgaccgaatgcagaagga

SEQ ID NO: 4 - DNA, 21 bp, synthetic construct
  source 1..21: mol_type = other DNA
acagagtatttgcgctccgaa

SEQ ID NO: 5 - DNA, 21 bp, synthetic construct
  source 1..21: mol_type = other DNA
atgtcgtccatcttgccattc

SEQ ID NO: 6 - DNA, 23 bp, synthetic construct
  source 1..23: mol_type = other DNA
aaccgtcctgttttctttagctt

SEQ ID NO: 7 - DNA, 21 bp, synthetic construct
  source 1..21: mol_type = other DNA
cccgaggttgtctggttcaaa

SEQ ID NO: 8 - DNA, 20 bp, synthetic construct
  source 1..20: mol_type = other DNA
gcaggtgtacttggcatcgt

SEQ ID NO: 9 - DNA, 19 bp, synthetic construct
  source 1..19: mol_type = other DNA
agccgaggttttaactgcg

SEQ ID NO: 10 - DNA, 20 bp, synthetic construct
  source 1..20: mol_type = other DNA
cccactcggtaagtgttccc

SEQ ID NO: 11 - DNA, 21 bp, synthetic construct
  source 1..21: mol_type = other DNA
gagggccaagacgaagacatc

SEQ ID NO: 12 - DNA, 21 bp, synthetic construct
  source 1..21: mol_type = other DNA
cagatcacgtcatcgcacaac

SEQ ID NO: 13 - DNA, 22 bp, synthetic construct
  source 1..22: mol_type = other DNA
tacctgaacccgtgttgctctc

SEQ ID NO: 14 - DNA, 22 bp, synthetic construct
  source 1..22: mol_type = other DNA
gttgctgaggtatcgccaggaa

SEQ ID NO: 15 - protein, 1401 aa, Homo sapiens
MAPQMYEFHLPLSPEELLKSGGVNQYVVQEVLSIKHLPPQLRAFQAAFRAQGPLAMLQHFDTIYSILHHFRSIDPGLKEDTLQFLIKVVSRHSQELPAILDDTTLSGSDRNAHLNALKMNCYALIRLLESFETMASQTNLVDLDLGGKGKKARTKAAHGFDWEEERQPILQLLTQLLQLDIRHLWNHSIIEEEFVSLVTGCCYRLLENPTINHQKNRPTREAITHLLGVALTRYNHMLSATVKIIQMLQHFEHLAPVLVAAVSLWATDYGMKSIVGEIVREIGQKCPQELSRDPSGTKGFAAFLTELAERVPAILMSSMCILLDHLDGENYMMRNAVLAAMAEMVLQVLSGDQLEAAARDTRDQFLDTLQAHGHDVNSFVRSRVLQLFTRIVQQKALPLTRFQAVVALAVGRLADKSVLVCKNAIQLLASFLANNPFSCKLSDADLAGPLQKETQKLQEMRAQRRTAAASAVLDPEEEWEAMLPELKSTLQQLLQLPQGEEEIPEQIANTETTEDVKGRIYQLLAKASYKKAIILTREATGHFQESEPFSHIDPEESEETRLLNILGLIFKGPAASTQEKNPRESTGNMVTGQTVCKNKPNMSDPEESRGNDELVKQEMLVQYLQDAYSFSRKITEAIGIISKMMYENTTTVVQEVIEFFVMVFQFGVPQALFGVRRMLPLIWSKEPGVREAVLNAYRQLYLNPKGDSARAKAQALIQNLSLLLVDASVGTIQCLEEILCEFVQKDELKPAVTQLLWERATEKVACCPLERCSSVMLLGMMARGKPEIVGSNLDTLVSIGLDEKFPQDYRLAQQVCHAIANISDRRKPSLGKRHPPFRLPQEHRLFERLRETVTKGFVHPDPLWIPFKEVAVTLIYQLAEGPEVICAQILQGCAKQALEKLEEKRTSQEDPKESPAMLPTFLLMNLLSLAGDVALQQLVHLEQAVSGELCRRRVLREEQEHKTKDPKEKNTSSETTMEEELGLVGATADDTEAELIRGICEMELLDGKQTLAAFVPLLLKVCNNPGLYSNPDLSAAASLALGKFCMISATFCDSQLRLLFTMLEKSPLPIVRSNLMVATGDLAIRFPNLVDPWTPHLYARLRDPAQQVRKTAGLVMTHLILKDMVKVKGQVSEMAVLLIDPEPQIAALAKNFFNELSHKGNAIYNLLPDIISRLSDPELGVEEEPFHTIMKQLLSYITKDKQTESLVEKLCQRFRTSRTERQQRDLAYCVSQLPLTERGLRKMLDNFDCFGDKLSDESIFSAFLSVVGKLRRGAKPEGKAIIDEFEQKLRACHTRGLDGIKELEIGQAGSQRAPSAKKPSTGSRYQPLASTASDNDFVTPEPRRTTRRHPNTQQRASKKKPKVVFSSDESSEEDLSAEMTEDETPKKTTPILRASARRHRS

SEQ ID NO: 16 - protein, 377 aa, Homo sapiens
MCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 17 - protein, 467 aa, Homo sapiens
MSSILPFTPPVVKRLLGWKKSAGGSGGAGGGEQNGQEEKWCEKAVKSLVKKLKKTGRLDELEKAITTQNCNTKCVTIPSTCSEIWGLSTPNTIDQWDTTGLYSFSEQTRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 18 - protein, 795 aa, Homo sapiens
MDTKLNMLNEKVDQLLHFQEDVTEKLQSMCRDMGHLERGLHRLEASRAPGPGGADGVPHIDTQAGWPEVLELVRAMQQDAAQHGARLEALFRMVAAVDRAIALVGATFQKSKVADFLMQGRVPWRRGSPGDSPEENKERVEEEGGKPKHVLSTSGVQSDAREPGEESQKADVLEGTAERLPPIRASGLGADPAQAVVSPGQGDGVPGPAQAFPGHLPLPTKVEAKAPETPSENLRTGLELAPAPGRVNVVSPSLEVAPGAGQGASSSRPDPEPLEEGTRLTPGPGPQCPGPPGLPAQARATHSGGETPPRISIHIQEMDTPGEMLMTGRGSLGPTLTTEAPAAAQPGKQGPPGTGRCLQAPGTEPGEQTPEGARELSPLQESSSPGGVKAEEEQRAGAEPGTRPSLARSDDNDHEVGALGLQQGKSPGAGNPEPEQDCAARAPVRAEAVRRMPPGAEAGSVVLDDSPAPPAPFEHRVVSVKETSISAGYEVCQHEVLGGGRFGQVHRCTEKSTGLPLAAKIIKVKSAKDREDVKNEINIMNQLSHVNLIQLYDAFESKHSCTLVMEYVDGGELFDRITDEKYHLTELDVVLFTRQICEGVHYLHQHYILHLDLKPENILCVNQTGHQIKIIDFGLARRYKPREKLKVNFGTPEFLAPEVVNYEFVSFPTDMWSVGVITYMLLSGLSPFLGETDAETMNFIVNCSWDFDADTFEGLSEEAKDFVSRLLVKEKSCRMSATQCLKHEWLNNLPAKASRSKTRLKSQLLLQKYIAQRKWKKHFYVVTAANRLRKFPTSP

SEQ ID NO: 19 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 20 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 21 - protein, 390 aa, synthetic construct
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS